(19) United States
(12) Patent Application Publication
(10) Pub. No.: US 2005/0221409 A1
(43) Pub. Date: Oct. 6, 2005

(54) Methods for the identification of inhibitors of amidophosphoribosyltransferase as antibiotics

(76) Inventors: Matthew M. Tanzer, Durham, NC (US); Jeffrey R. Shuster, Chapel Hill, NC (US); Lisbeth Hamer, Durham, NC (US); Kiichi Adachi, Osaka (JP); Todd M. DeZwaan, Apex, NC (US); Sze-Chung C. Lo, Hong Kong (CN); Maria Victoria Montenegro-Chamorro, Durham, NC (US); Blaise A. Darveaux, Hillsborough, NC (US); Sheryl A. Frank, Durham, NC (US); Ryan W. Heiniger, Holly Springs, NC (US); Sanjoy K. Mahanty, Chapel Hill, NC (US); Huaqin Pan, Apex, NC (US); Amy S. Covington, Raleigh, NC (US); Rex Tarpey, Apex, NC (US)

Correspondence Address: ERIC J. KRON;ICORIA, INC., 108 T.W. ALEXANDER DRIVE, BUILDING 1A, POST OFFICE BOX 14528, RESEARCH TRIANGLE PARK, NC, 27709, US

(21) Appl. No.: 10/969,709

(22) Filed: Oct. 20, 2004

Related U.S. Application Data
(60) Provisional application No. 60/513,829, filed on Oct. 23, 2003.

(51) Int. Cl. C12Q 1/48; C12Q 1/18

(52) U.S. Cl. 435/15; 435/32

(57) ABSTRACT

The present inventors have discovered that amidophosphoribosyltransferase is essential for normal fungal pathogenicity. Specifically, the inhibition of amidophosphoribosyltransferase gene expression in fungi results in drastically reduced pathogenicity. Thus, amidophosphoribosyltransferase can be used as a target for the identification of antibiotics, preferably antifungals. Accordingly, the present invention provides methods for the identification of compounds that inhibit amidophosphoribosyltransferase expression or activity. The methods of the invention are useful for the identification of antibiotics, preferably antifungals.

The present application claims the benefit of U.S. Application Ser. No. 60/513,829 filed on Oct. 23, 2003, herein incorporated by reference in its entirety.

### FIELD OF THE INVENTION

The invention relates generally to methods for the identification of antibiotics, preferably antifungals that affect the biosynthesis of purine.

### BACKGROUND OF THE INVENTION

Filamentous fungi are causal agents responsible for many serious pathogenic infections of plants and animals. Since fungi are eukaryotes, and thus more similar to their host organisms than, for example bacteria, the treatment of infections by fungi poses special risks and challenges not encountered with other types of infections. One such fungus is Magnaporthe grisea, the fungus that causes rice blast disease, a significant threat to food supplies worldwide. Other examples of plant pathogens of economic importance include the pathogens in the genera Agaricus, Alternaria, Anisogramma, Anthracoidea, Antrodia, Apiognomonia, Apiosporina, Armillaria, Ascochyta, Aspergillus, Bipolaris, Bjerkandera, Botryosphaeria, Botrytis, Ceratobasidium, Ceratocystis, Cercospora, Cercosporidium, Cerotelium, Cerrena, Chondrostereum, Chryphonectria, Chrysomyxa, Cladosporium, Claviceps, Cochliobolus, Coleosporium, Colletotrichium, Colletotrichum, Corticium, Corynespora, Cronartium, Cryphonectria, Cryptosphaeria, Cyathus, Cymadothea, Cytospora, Daedaleopsis, Diaporthe, Didymella, Diplocarpon, Diplodia, Discohainesia, Discula, Dothistroma, Drechslera, Echinodontium, Elsinoe, Endocronartium, Endothia, Entyloma, Epichloe, Erysiphe, Exobasidium, Exserohilum, Fomes, Fomitopsis, Fusarium, Gaeumannomyces, Ganoderma, Gibberella, Gloeocercospora, Gloeophyllum, Gloeoporus, Glomerella, Gnomoniella, Guignardia, Gymnosporangium, Helminthosporium, Herpotrichia, Heterobasidion, Hirschioporus, Hypodermella, Inonotus, Irpex, Kabatiella, Kabatina, Laetiporus, Laetisaria, Lasiodiplodia, Laxitextum, Leptographium, Leptosphaeria, Leptosphaerulina, Leucytospora, Linospora, Lophodermella, Lophodermium, Macrophomina, Magnaporthe, Marssonina, Melampsora, Melampsorella, Meria, Microdochium, Microsphaera, Monilinia, Monochaetia, Morchella, Mycosphaerella, Myrothecium, Nectria, Nigrospora, Ophiosphaerella, Ophiostoma, Penicillium, Perenniporia, Peridermium, Pestalotia, Phaeocryptopus, Phaeolus, Phakopsora, Phellinus, Phialophora, Phoma, Phomopsis, Phragmidium, Phyllachora, Phyllactinia, Phyllosticta, Phymatotrichopsis, Pleospora, Podosphaera, Pseudopeziza, Pseudoseptoria, Puccinia, Pucciniastrum, Pyricularia, Rhabdocline, Rhizoctonia, Rhizopus, Rhizosphaera, Rhynchosporium, Rhytisma, Schizophyllum, Schizopora, Scirrhia, Sclerotinia, Sclerotium, Scytinostroma, Septoria, Setosphaera, Sirococcus, Spaerotheca, Sphaeropsis, Sphaerotheca, Sporisorium, Stagonospora, Stemphylium, Stenocarpella, Stereum, Taphrina, Thielaviopsis, Tilletia, Trametes, Tranzschelia, Trichoderma, Tubakia, Typhula, Uncinula, Urocystis, Uromyces, Ustilago, Valsa, Venturia, Verticillium, Xylaria, and others. Related organisms are classified in the oomycetes classification and include the genera Albugo, Aphanomyces, Bremia, Peronospora, Phytophthora, Plasmodiophora, Plasmopara, Pseudoperonospora, Pythium, Sclerophthora, and others. Oomycetes are also significant plant pathogens and are sometimes classified along with the true fungi. Human diseases that are caused by filamentous fungi include life-threatening lung and disseminated diseases, often a result of infections by Aspergillus fumigatus. Other fungal diseases in animals are caused by fungi in the genera Fusarium, Blastomyces, Microsporum, Trichophyton, Epidermophyton, Candida, Histoplamsa, Pneumocystis, Cryptococcus, other Aspergilli, and others. The control of fungal diseases in plants and animals is usually mediated by chemicals that inhibit the growth, proliferation, and/or pathogenicity of the fungal organisms. To date, there are less than twenty known modes-of-action for plant protection fungicides and human antifungal compounds.

A pathogenic organism has been defined as an organism that causes, or is capable of causing disease. Pathogenic organisms propagate on or in tissues and may obtain nutrients and other essential materials from their hosts. A substantial amount of work concerning filamentous fungal pathogens has been performed with the human pathogen, Aspergillus fumigatus. Shibuya et al., 27 Microb. Pathog. 123 (1999) (PubMed Identifier (PMID): 10455003) have shown that the deletion of either of two suspected pathogenicity related genes encoding an alkaline protease or a hydrophobin (rodlet), respectively, did not reduce mortality of mice infected with these mutant strains. Smith et al., 62 Infect. Immun. 5247 (1994) (PMID: 7960101) showed similar results with alkaline protease and the ribotoxin restrictocin; Aspergillus fumigatus strains mutated for either of these genes were fully pathogenic to mice. Reichard et al., 35 J. Med. Vet. Mycol. 189 (1997) (PMID: 9229335)) showed that deletion of the suspected pathogenicity gene encoding aspergillopepsin (PEP) in Aspergillus fumigatus had no effect on mortality in a guinea pig model system, whereas Aufauvre-Brown et al., 21 Fungal. Genet. Biol. 141 (1997) (PMID: 9073488) showed no effects of a chitin synthase mutation on pathogenicity.

However, not all experiments produced negative results. Ergosterol is an important membrane component found in fungal organisms. Pathogenic fungi lacking key enzymes in the ergosterol biochemical pathway might be expected to be non-pathogenic since neither the plant nor animal hosts contain this particular sterol. Many antifungal compounds that affect the ergosterol biochemical pathway have been previously described. (U.S. Pat. Nos. 4,920,109; 4,920,111; 4,920,112; 4,920,113; and 4,921,844; Hewitt, H. G. Fungicides in Crop Protection Cambridge, University Press (1998)). D'Enfert et al., 64 Infect. Immun. 4401 (1996) (PMID: 8926121)) showed that an Aspergillus fumigatus strain mutated in an orotidine 5′-phosphate decarboxylase gene was entirely non-pathogenic in mice, and Brown et al. (Brown et al., 36 Mol. Microbiol. 1371 (2000) (PMID: 10931287)) observed a non-pathogenic result when genes involved in the synthesis of para-aminobenzoic acid were mutated. Some specific target genes have been described as having utility for the screening of inhibitors of plant pathogenic fungi. U.S. Pat. No. 6,074,830 to Bacot et al., describe the use of 3,4-dihydroxy-2-butanone 4-phosphate synthase, and U.S. Pat. No. 5,976,848 to Davis et al. describes the use of dihydroorotate dehydrogenase for potential screening purposes.

There are also a number of papers that report less clear results, showing neither full pathogenicity nor non-pathogenicity of mutants. For example, Hensel et al. (Hensel, M. et al., 258 Mol. Gen. Genet. 553 (1998) (PMID: 9669338)) showed only moderate effects of the deletion of the area transcriptional activator on the pathogenicity of Aspergillus fumigatus. Therefore, it is not currently possible to determine which specific growth materials may be readily obtained by a pathogen from its host, and which materials may not.

The present invention discloses amidophosphoribosyltransferase as a target for the identification of antifungal, biocide, and biostatic materials.

### SUMMARY OF THE INVENTION

The present inventors have discovered that in vivo disruption of the gene encoding amidophosphoribosyltransferase (ADE4) in Magnaporthe grisea severely reduces the pathogenicity of the fungus. Thus, the present inventors have discovered that amidophosphoribosyltransferase is useful as a target for the identification of antibiotics, preferably fungicides. Accordingly, the present invention provides methods for the identification of compounds that inhibit amidophosphoribosyltransferase expression or activity. The methods of the invention are useful for the identification of antibiotics, preferably fungicides.

### BRIEF DESCRIPTION OF THE FIGURES

FIG. 1. Diagram of the reversible reaction catalyzed by amidophosphoribosyltransferase (ADE4). The enzyme catalyzes the reversible interconversion of 5-phospho-beta-D-ribosylamine, diphosphate, and L-glutamate to 5-phospho-alpha-D-ribose 1-diphosphate and L-glutamine. This reaction is part of the purine biosynthesis pathway.

FIG. 2. Digital image showing the effect of ADE4 gene disruption on Magnaporthe grisea pathogenicity using whole plant infection assays. Rice variety CO₃₉ was inoculated with wild-type strain Guy11, transposon insertion strains, K1-4, and K1-8. Leaf segments were imaged at seven days post-inoculation.

FIGS. 3A and 3B. The wild type and two mutant strains were subjected to nutritional profiling by inoculating conidial suspensions into 96-well auxotrophy plates. FIG. 3A shows greatly reduced growth of transformants, as compared to wildtype, on adenine-deficient medium. FIG. 3B shows restored growth when adenine is added to the medium. T1 and T2 correspond to K1-4 and K1-8, respectively. The y-axes in 3A and 3B represent turbidity, measured by OD₄₉₀+OD₇₅₀. The OD₄₉₀ measures the extent of tetrazolium dye reduction and the level of growth, and OD₇₅₀ measures growth only.

### DETAILED DESCRIPTION OF THE INVENTION

Unless otherwise indicated, the following terms are intended to have the following meanings in interpreting the present invention.

As used herein, the term “ADE4” means a gene encoding amidophosphoribosyltransferase activity, referring to an enzyme that catalyses the reversible interconversion of 5-phospho-beta-D-ribosylamine, diphosphate, and L-glutamate to 5-phospho-alpha-D-ribose 1-diphosphate and L-glutamine. ADE4 or amidophosphoribosyltransferase is also used used herein to refer to the amidophosphoribosyltransferase polypeptide. By “fungal ADE4” or “fungal amidophosphoribosyltransferase” is meant an enzyme that can be found in at least one fungus, and that catalyzes the reversible interconversion of 5-phospho-beta-D-ribosylamine, diphosphate, and L-glutamate to 5-phospho-alpha-D-ribose 1-diphosphate and L-glutamine.

As used herein, the terms “amidophosphoribosyltransferase” and “amidophosphoribosyltransferase polypeptide” are synonymous with “the ADE4 gene product” and refer to an enzyme that catalyzes the reversible interconversion of 5-phospho-beta-D-ribosylamine, diphosphate, and L-glutamate to 5-phospho-alpha-D-ribose 1-diphosphate and L-glutamine.

The term “antibiotic” refers to any substance or compound that when contacted with a living cell, organism, virus, or other entity capable of replication, results in a reduction of growth, viability, or pathogenicity of that entity.

The term “antipathogenic”, as used herein, refers to a mutant form of a gene, which inactivates a pathogenic activity of an organism on its host organism or substantially reduces the level of pathogenic activity, wherein “substantially” means a reduction at least as great as the standard deviation for a measurement, preferably a reduction by 50%, more preferably a reduction of at least one magnitude, i.e. to 10%. The pathogenic activity affected may be an aspect of pathogenic activity governed by the normal form of the gene, or the pathway the normal form of the gene functions on, or the organism's pathogenic activity in general. “Antipathogenic” may also refer to a cell, cells, tissue, or organism that contains the mutant form of a gene; a phenotype associated with the mutant form of a gene, and/or associated with a cell, cells, tissue, or organism that contain the mutant form of a gene.

The term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions and hydrophobic interactions among nonpolar groups. One or more of these interactions can mediate the binding of two molecules to each other.

The term “biochemical pathway” or “pathway” refers to a connected series of biochemical reactions normally occurring in a cell, or more broadly a cellular event such as cellular division or DNA replication. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent, may, but does not necessarily, act directly on the expression product of that particular gene.

As used herein, the term “conditional lethal” refers to a mutation permitting growth and/or survival only under special growth or environmental conditions.

As used herein, the term “cosmid” refers to a hybrid vector, used in gene cloning, that includes a cos site (from the lambda bacteriophage). In some cases, the cosmids of the invention comprise drug resistance marker genes and other plasmid genes. Cosmids are especially suitable for cloning large genes or multigene fragments.

“Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia, perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. They can either exist as single cells or make up a multicellular body called a mycelium, which consists of filaments known as hyphae. Most fungal cells are multinucleate and have cell walls, composed chiefly of chitin. Fungi exist primarily in damp situations on land and, because of the absence of chlorophyll and thus the inability to manufacture their own food by photosynthesis, are either parasites on other organisms or saprotrophs feeding on dead organic matter. The principal criteria used in classification are the nature of the spores produced and the presence or absence of cross walls within the hyphae. Fungi are distributed worldwide in terrestrial, freshwater, and marine habitats. Some live in the soil. Many pathogenic fungi cause disease in animals and man or in plants, while some saprotrophs are destructive to timber, textiles, and other materials. Some fungi form associations with other organisms, most notably with algae to form lichens.

As used herein, the term “fungicide,” “antifungal,” or “antimycotic” refers to an antibiotic substance or compound that kills or suppresses the growth, viability, or pathogenicity of at least one fungus, fungal cell, fungal tissue or spore.

In the context of this disclosure, “gene” should be understood to refer to a unit of heredity. Each gene is composed of a linear chain of deoxyribonucleotides that can be referred to by the sequence of nucleotides forming the chain. Thus, “sequence” is used to indicate both the ordered listing of the nucleotides which form the chain, and the chain, itself, which has that sequence of nucleotides. “Sequence” is used in the similar way in referring to RNA chains, linear chains made of ribonucleotides. The gene may include regulatory and control sequences, sequences which can be transcribed into an RNA molecule, and may contain sequences with unknown function. The majority of the RNA transcription products are messenger RNAs (mRNAs), which include sequences which are translated into polypeptides and may include sequences which are not translated. It should be recognized that small differences in nucleotide sequence for the same gene can exist between different fungal strains, or even within a particular fungal strain, without altering the identity of the gene.

As used in this disclosure, the terms “growth” or “cell growth” of an organism refer to an increase in mass, density, or number of cells of the organism. Some common methods for the measurement of growth include the determination of the optical density of a cell suspension, the counting of the number of cells in a fixed volume, the counting of the number of cells by measurement of cell division, the measurement of cellular mass or cellular volume, and the like.

As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used herein, the term “heterologous amidophosphoribosyltransferase” or “heterologous ADE4” means either a nucleic acid encoding a polypeptide or a polypeptide, wherein the polypeptide has at least 50%, 55%, 60%, 65%, 70%, 75%, 80%, 85%, 90%, 91%, 92%, 93%, 94%, 95%, 96%, 97%, 98%, or 99% sequence identity or each integer unit of sequence identity from 50-100% in ascending order to M. grisea amidophosphoribosyltransferase protein (SEQ ID NO:3) and at least 10%, 25%, 50%, 75%, 80%, 90%, 95%, or 99% activity or each integer unit of activity from 10-100% in ascending order of the activity of M. grisea amidophosphoribosyltransferase protein (SEQ ID NO:3). Examples of heterologous amidophosphoribosyltransferases include, but are not limited to, amidophosphoribosyltransferase from Schizosaccharomyces pombe, amidophosphoribosyltransferase from Saccharomyces kluyveri, and amidophosphoribosyltransferase from Saccharomyces cerevisiae.

As used herein, the term “His-Tag” refers to an encoded polypeptide consisting of multiple consecutive histidine amino acids.

As used herein, the terms “hph,” “hygromycin B phosphotransferase,” and “hygromycin resistance gene” refer to a hygromycin phosphotransferase gene or gene product.

As used herein, the term “imperfect state” refers to a classification of a fungal organism having no demonstrable sexual life stage.

The term “inhibitor,” as used herein, refers to a chemical substance that inactivates the enzymatic activity of amidophosphoribosyltransferase or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide may be “introduced” into a fungal cell by any means known to those of skill in the art, including transfection, transformation or transduction, transposable element, electroporation, particle bombardment, infection and the like. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active.

As used herein, the term “knockout” or “gene disruption” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker.

As used herein, the term “mutant form” of a gene refers to a gene which has been altered, either naturally or artificially, changing the base sequence of the gene. The change in the base sequence may be of several different types, including changes of one or more bases for different bases, deletions, and/or insertions, such as by a transposon. In contrast, a normal form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

The term “NAD(P)” is herein used to mean either “NAD” or “NADP” and, similarly, the term “NAD(P)H” is herein used to mean “NADH” or “NADPH.”

As used herein, the term “Ni-NTA” refers to nickel sepharose.

As used herein, a “normal” form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “one form” of a gene is synonymous with the term “gene,” and a “different form” of a gene refers to a gene that has greater than 49% sequence identity and less than 100% sequence identity with the first form.

As used herein, the term “pathogenicity” refers to a capability of causing disease and/or degree of capacity to cause disease. The term is applied to parasitic micro-organisms in relation to their hosts. As used herein, “pathogenicity,” “pathogenic,” and the like, encompass the general capability of causing disease as well as various mechanisms and structural and/or functional deviations from normal used in the art to describe the causative factors and/or mechanisms, presence, pathology, and/or progress of disease, such as virulence, host recognition, cell wall degradation, toxin production, infection hyphae, penetration peg production, appressorium production, lesion formation, sporulation, and the like.

The “percent (%) sequence identity” between two polynucleotide or two polypeptide sequences is determined according to either the BLAST program (Basic Local Alignment Search Tool; (Altschul, S. F. et al., 215 J. Mol. Biol. 403 (1990) (PMID: 2231712)) or using Smith Waterman Alignment (T. F. Smith & M. S. Waterman (1981) 147 J. Mol. Biol. 195 (1981) (PMID: 7265238)). It is understood that for the purposes of determining sequence identity when comparing a DNA sequence to an RNA sequence, a thymine nucleotide is equivalent to a uracil nucleotide.

By “polypeptide” is meant a chain of at least two amino acids joined by peptide bonds. The chain may be linear, branched, circular or combinations thereof. The polypeptides may contain amino acid analogs and other modifications, including, but not limited to glycosylated or phosphorylated residues.

As used herein, the term “proliferation” is synonymous to the term “growth.”

As used herein, “semi-permissive conditions” are conditions in which the relevant culture parameter for a particular growth conditional phenotype is intermediate between permissive conditions and non-permissive conditions. Consequently, in semi-permissive conditions an organism having a growth conditional phenotype will exhibit growth rates intermediate between those shown in permissive conditions and non-permissive conditions. In general, such intermediate growth rate may be due to a mutant cellular component which is partially functional under semi-permissive conditions, essentially fully functional under permissive conditions, and is non-functional or has very low function under non-permissive conditions, where the level of function of that component is related to the growth rate of the organism. An intermediate growth rate may also be a result of a nutrient substance or substances that are present in amounts not sufficient for optimal growth rates to be achieved.

“Sensitivity phenotype” refers to a phenotype that exhibits either hypersensitivity or hyposensitivity.

The term “specific binding” refers to an interaction between amidophosphoribosyltransferase and a molecule or compound, wherein the interaction is dependent upon the primary amino acid sequence and/or the tertiary conformation of amidophosphoribosyltransferase. An “amidophosphoribosyltransferase ligand” is an example of specific binding.

“Transform,” as used herein, refers to the introduction of a polynucleotide (single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. This process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof which retain the polynucleotide of interest.

For the purposes of the invention, “transgenic” refers to any cell, spore, tissue or part, that contains all or part of at least one recombinant polynucleotide. In many cases, all or part of the recombinant polynucleotide is stably integrated into a chromosome or stable extra-chromosomal element, so that it is passed on to successive generations.

As used herein, the term “Tween 20” means sorbitan mono-9-octadecenoate poly(oxy-1,1-ethanediyl).

As used in this disclosure, the term “viability” of an organism refers to the ability of an organism to demonstrate growth under conditions appropriate for the organism, or to demonstrate an active cellular function. Some examples of active cellular functions include respiration as measured by gas evolution, secretion of proteins and/or other compounds, dye exclusion, mobility, dye oxidation, dye reduction, pigment production, changes in medium acidity, and the like.

The present inventors have discovered that disruption of the ADE4 gene and/or gene product reduces the pathogenicity of Magnaporthe grisea. Thus, the inventors are the first to demonstrate that amidophosphoribosyltransferase is a target for antibiotics, preferably antifungals.

Accordingly, the invention provides methods for identifying compounds that inhibit ADE4 gene expression or biological activity of its gene product(s). Such methods include ligand-binding assays, assays for enzyme activity, cell-based assays, and assays for ADE4 gene expression. The compounds identified by the methods of the invention are useful as antibiotics.

Thus, in one embodiment, the invention provides a method for identifying a test compound as a candidate for an antibiotic, comprising contacting an amidophosphoribosyltransferase polypeptide with a test compound and detecting the presence or absence of binding between the test compound and the amidophosphoribosyltransferase polypeptide, wherein binding indicates that the test compound is a candidate for an antibiotic.

The amidophosphoribosyltransferase polypeptides of the invention have the amino acid sequence of a naturally occurring amidophosphoribosyltransferase found in a fungus, animal, plant, or microorganism, or have an amino acid sequence derived from a naturally occurring sequence. Preferably the amidophosphoribosyltransferase is a fungal amidophosphoribosyltransferase. A cDNA encoding M. grisea amidophosphoribosyltransferase protein is set forth in SEQ ID NO:1, an M. grisea ADE4 genomic DNA is set forth in SEQ ID NO:2, and an M. grisea amidophosphoribosyltransferase polypeptide is set forth in SEQ ID NO:3. In one embodiment, the amidophosphoribosyltransferase is a Magnaporthe amidophosphoribosyltransferase. Magnaporthe species include, but are not limited to, Magnaporthe rhizophila, Magnaporthe salvinii, Magnaporthe grisea and Magnaporthe poae and the imperfect states of Magnaporthe in the genus Pyricularia. Preferably, the Magnaporthe amidophosphoribosyltransferase is from Magnaporthe grisea.

In one embodiment, the invention provides a polypeptide consisting essentially of SEQ ID NO:3. For the purposes of the present invention, a polypeptide consisting essentially of SEQ ID NO:3 has at least 50% sequence identity with M. grisea amidophosphoribosyltransferase (SEQ ID NO:3) and at least 10% of the activity of SEQ ID NO:3. A polypeptide consisting essentially of SEQ ID NO:3 has at least 91%, 92%, 93%, 94%, 95%, 96%, 97%, 98%, or 99% sequence identity with SEQ ID NO:3 and at least 25%, 50%, 75%, or 90% of the activity of M. grisea amidophosphoribosyltransferase.

In various embodiments, the amidophosphoribosyltransferase can be from Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), and the like.

Fragments of an amidophosphoribosyltransferase polypeptide are useful in the methods of the invention. In one embodiment, the amidophosphoribosyltransferase fragments include an intact or nearly intact epitope that occurs on the biologically active wild-type amidophosphoribosyltransferase. The fragments comprise at least 10 consecutive amino acids of an amidophosphoribosyltransferase. The fragments comprises at least 15, 20, 25, 30, 35, 40, 50, 60, 70, 80, 90, 100, 125, 150, 175, 200, 225, 250, 300, 325, 350, 375, 400, 425, 450, 475, 500, 525, or at least 537 consecutive amino acid residues of an amidophosphoribosyltransferase. In one embodiment, the fragment is from a Magnaporthe amidophosphoribosyltransferase. In one embodiment, the fragment contains an amino acid sequence conserved among fungal amidophosphoribosyltransferases.

Polypeptides having at least 50% sequence identity with M. grisea amidophosphoribosyltransferase (SEQ ID NO:3) protein are also useful in the methods of the invention. In one embodiment, the sequence identity is at least 55%, 60%, 65%, 70%, 75%, 80%, 85%, 90%, 91%, 92%, 93%, 94%, 95%, 96%, 97%, 98% or 99%, or any integer from 50-100% sequence identity in ascending order with M. grisea amidophosphoribosyltransferase (SEQ ID NO:3) protein. In addition, it is preferred that polypeptides of the invention have at least 10% of the activity of M. grisea amidophosphoribosyltransferase (SEQ ID NO:3) protein. Amidophosphoribosyltransferase polypeptides of the invention have at least 10%, 15%, 20%, 25%, 30%, 35%, 40%, 45%, 50%, 55%, 60%, 65%, 70%, 75%, 80%, 85% or at least 90% of the activity of M. grisea amidophosphoribosyltransferase (SEQ ID NO:3) protein.

Thus, in another embodiment, the invention provides a method for identifying a test compound as a candidate for a fungicide, comprising: contacting a test compound with at least one polypeptide selected from the group consisting of: a polypeptide consisting essentially of SEQ ID NO:3, a polypeptide having at least ten consecutive amino acids of an M. grisea amidophosphoribosyltransferase (SEQ ID NO:3) protein, a polypeptide having at least 50% sequence identity with an M. grisea amidophosphoribosyltransferase (SEQ ID NO: 3) protein and at least 10% of the activity of an M. grisea amidophosphoribosyltransferase (SEQ ID NO:3) protein; and a polypeptide consisting of at least 50 amino acids having at least 50% sequence identity with an M. grisea amidophosphoribosyltransferase (SEQ ID NO:3) protein and at least 10% of the activity of an M. grisea amidophosphoribosyltransferase (SEQ ID NO:3) protein; and detecting the presence and/or absence of binding between the test compound and the polypeptide, wherein binding indicates that the test compound is a candidate for an antibiotic.

Any technique for detecting the binding of a ligand to its target may be used in the methods of the invention. For example, the ligand and target are combined in a buffer. Many methods for detecting the binding of a ligand to its target are known in the art, and include, but are not limited to, the detection of an immobilized ligand-target complex or the detection of a change in the properties of a target when it is bound to a ligand. For example, in one embodiment, an array of immobilized candidate ligands is provided. The immobilized ligands are contacted with an amidophosphoribosyltransferase protein or a fragment or variant thereof, the unbound protein is removed and the bound amidophosphoribosyltransferase is detected. In a preferred embodiment, bound amidophosphoribosyltransferase is detected using a labeled binding partner, such as a labeled antibody. In an alternate preferred embodiment, amidophosphoribosyltransferase is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for an antibiotic, it can be tested for the ability to inhibit amidophosphoribosyltransferase enzymatic activity. The compounds can be tested using either in vitro or cell based assays. Alternatively, a compound can be tested by applying it directly to a fungus or fungal cell, or expressing it therein, and monitoring the fungus or fungal cell for changes or decreases in growth, development, viability, pathogenicity, or alterations in gene expression. Thus, in one embodiment, the invention provides a method for determining whether a compound identified as an antibiotic candidate by an above method has antifungal activity, further comprising: contacting a fungus or fungal cells with the antifungal candidate and detecting a decrease in the growth, viability, or pathogenicity of the fungus or fungal cells.

By decrease in growth, is meant that the antifungal candidate causes at least a 10% decrease in the growth of the fungus or fungal cells, as compared to the growth of the fungus or fungal cells in the absence of the antifungal candidate. By a decrease in viability is meant that at least 20% of the fungal cells, or portion of the fungus contacted with the antifungal candidate are nonviable. Preferably, the growth or viability will be decreased by at least 40%. More preferably, the growth or viability will be decreased by at least 50%, 75% or at least 90% or more. Methods for measuring fungal growth and cell viability are known to those skilled in the art. By decrease in pathogenicity, is meant that the antifungal candidate causes at least a 10% decrease in the disease caused by contact of the fungal pathogen with its host, as compared to the disease caused in the absence of the antifungal candidate. Preferably, the disease will be decreased by at least 40%. More preferably, the disease will be decreased by at least 50%, 75% or at least 90% or more. Methods for measuring fungal disease are well known to those skilled in the art, and include such metrics as lesion formation, lesion size, sporulation, respiratory failure, and/or death.

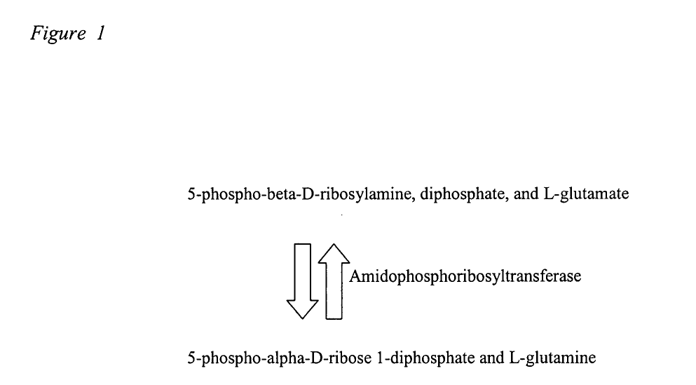

The ability of a compound to inhibit amidophosphoribosyltransferase activity can be detected using in vitro enzymatic assays in which the disappearance of a substrate or the appearance of a product is directly or indirectly detected. Amidophosphoribosyltransferase catalyzes the reversible interconversion of 5-phospho-beta-D-ribosylamine, diphosphate, and L-glutamate to 5-phospho-alpha-D-ribose 1-diphosphate and L-glutamine (see FIG. 1). Methods for detection of 5-phospho-beta-D-ribosylamine, diphosphate, L-glutamate, 5-phospho-alpha-D-ribose 1-diphosphate and/or L-glutamine include spectrophotometry, fluorimetry, mass spectroscopy, thin layer chromatography (TLC) and reverse phase HPLC.

Thus, the invention provides a method for identifying a test compound as a candidate for an antibiotic, comprising: contacting 5-phospho-beta-D-ribosylamine, diphosphate and L-glutamate with an amidophosphoribosyltransferase in the presence and absence of a test compound or contacting 5-phospho-alpha-D-ribose 1-diphosphate and L-glutamine with an amidophosphoribosyltransferase in the presence and absence of a test compound; and determining a concentration for at least one of 5-phospho-beta-D-ribosylamine, diphosphate, L-glutamate, 5-phospho-alpha-D-ribose 1-diphosphate, and/or L-glutamine in the presence and absence of the test compound, wherein a change in the concentration for any of the above substances indicates that the test compound is a candidate for an antibiotic. Enzymatically active fragments of M. grisea amidophosphoribosyltransferase set forth in SEQ ID NO:3 are also useful in the methods of the invention. For example, an enzymatically active polypeptide comprising at least 50 consecutive amino acid residues and at least 10% of the activity of M. grisea amidophosphoribosyltransferase set forth in SEQ ID NO:3 are useful in the methods of the invention. In addition, enzymatically active polypeptides having at least 10% of the activity of SEQ ID NO:3 and at least 50%, 60%, 70%, 80%, 90%, 95%, 96%, 97%, 98% or 99% sequence identity with SEQ ID NO:3 are useful in the methods of the invention. Most preferably, the enzymatically active polypeptide has at least 50% sequence identity with SEQ ID NO:3 and at least 25%, 75% or at least 90% of the activity thereof.

Thus, the invention provides a method for identifying a test compound as a candidate for an antibiotic, comprising: contacting 5-phospho-beta-D-ribosylamine, diphosphate and L-glutamate or 5-phospho-alpha-D-ribose 1-diphosphate and L-glutamine with a polypeptide selected from the group consisting of: a polypeptide consisting essentially of SEQ ID NO:3, a polypeptide having at least 50% sequence identity with the M. grisea amidophosphoribosyltransferase set forth in SEQ ID NO:3 and having at least 10% of the activity thereof, a polypeptide comprising at least 50 consecutive amino acids of M. grisea amidophosphoribosyltransferase set forth in SEQ ID NO:3 and having at least 10% of the activity thereof, and a polypeptide consisting of at least 50 amino acids and having at least 50% sequence identity with M. grisea amidophosphoribosyltransferase set forth in SEQ ID NO:3 and having at least 10% of the activity thereof; contacting 5-phospho-beta-D-ribosylamine, diphosphate and L-glutamate or 5-phospho-alpha-D-ribose 1-diphosphate and L-glutamine with the polypeptide and a test compound; and determining a concentration for at least one of 5-phospho-beta-D-ribosylamine, diphosphate, L-glutamate, 5-phospho-alpha-D-ribose 1-diphosphate, and/or L-glutamine in the presence and absence of the test compound, wherein a change in concentration for any of the above substances indicates that the test compound is a candidate for an antibiotic.

For the in vitro enzymatic assays, amidophosphoribosyltransferase protein and derivatives thereof may be purified from a fungus or may be recombinantly produced in and purified from an archael, bacterial, fungal, or other eukaryotic cell culture. Preferably these proteins are produced using an E. coli, yeast, or filamentous fungal expression system. An example of a method for the purification of an amidophosphoribosyltransferase polypeptide is described in Tso et al. (1982) J Biol Chem 257:3532-3536. Other methods for the purification of amidophosphoribosyltransferase proteins and polypeptides are known to those skilled in the art.

As an alternative to in vitro assays, the invention also provides cell based assays. In one embodiment, the invention provides a method for identifying a test compound as a candidate for an antibiotic, comprising: a) measuring the expression or activity of an amidophosphoribosyltransferase in a cell, cells, tissue, or an organism in the absence of a test compound; b) contacting the cell, cells, tissue, or organism with the test compound and measuring the expression or activity of the amidophosphoribosyltransferase in the cell, cells, tissue, or organism; and c) comparing the expression or activity of the amidophosphoribosyltransferase in steps (a) and (b), wherein an altered expression or activity in the presence of the test compound indicates that the compound is a candidate for an antibiotic.

Expression of amidophosphoribosyltransferase can be measured by detecting the amidophosphoribosyltransferase primary transcript or mRNA, amidophosphoribosyltransferase polypeptide, or amidophosphoribosyltransferase enzymatic activity. Methods for detecting the expression of RNA and proteins are known to those skilled in the art. (See, e.g., Current Protocols in Molecular Biology, Ausubel et al., eds., Greene Publishing & Wiley-Interscience, New York, (1995)). The method of detection is not critical to the present invention. Methods for detecting amidophosphoribosyltransferase RNA include, but are not limited to amplification assays such as quantitative reverse transcriptase-PCR, and/or hybridization assays such as Northern analysis, dot blots, slot blots, in-situ hybridization, transcriptional fusions using an amidophosphoribosyltransferase promoter fused to a reporter gene, DNA assays, and microarray assays.

Methods for detecting protein expression include, but are not limited to, immunodetection methods such as Western blots, ELISA assays, polyacrylamide gel electrophoresis, mass spectroscopy, and enzymatic assays. Also, any reporter gene system may be used to detect amidophosphoribosyltransferase protein expression. For detection using gene reporter systems, a polynucleotide encoding a reporter protein is fused in frame with amidophosphoribosyltransferase, so as to produce a chimeric polypeptide. Methods for using reporter systems are known to those skilled in the art.

Chemicals, compounds or compositions identified by the above methods as modulators of amidophosphoribosyltransferase expression or activity can then be used to control fungal growth. Diseases such as rusts, mildews, and blights spread rapidly once established. Fungicides are thus routinely applied to growing and stored crops as a preventive measure, generally as foliar sprays or seed dressings. For example, compounds that inhibit fungal growth can be applied to a fungus or expressed in a fungus, in order to prevent fungal growth. Thus, the invention provides a method for inhibiting fungal growth, comprising contacting a fungus with a compound identified by the methods of the invention as having antifungal activity.

Antifungals and antifungal inhibitor candidates identified by the methods of the invention can be used to control the growth of undesired fungi, including ascomycota, zygomycota, basidiomycota, chytridiomycota, and lichens. Examples of undesired fungi include, but are not limited to Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), diseases of animals such as infections of lungs, blood, brain, skin, scalp, nails or other tissues (Aspergillus fumigatus Aspergillus sp. Fusraium sp., Trichophyton sp., Epidermophyton sp., and Microsporum sp., and the like).

Also provided in the invention are methods of screening for an antibiotic by determining the in vivo activity of a test compound against two separate fungal organisms, wherein the fungal organisms comprise a first form of an amidophosphoribosyltransferase and a second form of the amidophosphoribosyltransferase, respectively. In the methods of the invention, at least one of the two forms of the amidophosphoribosyltransferase has at least 10% of the activity of the polypeptide set forth in SEQ ID NO:3. The methods comprise comparing the growth of the two organisms in the presence of the test compound relative to their respective controls without test compound. A difference in growth between the two organisms in the presence of the test compound indicates that the test compound is a candidate for an antibiotic.

The forms of an amidophosphoribosyltransferase useful in the methods of the invention are selected from the group consisting of: a nucleic acid encoding SEQ ID NO:3, a nucleic acid encoding a polypeptide consisting essentially of SEQ ID NO:3, SEQ ID NO:1 or SEQ ID NO:2, SEQ ID NO:1 or SEQ ID NO:2 comprising a mutation either reducing or abolishing amidophosphoribosyltransferase protein activity, a heterologous amidophosphoribosyltransferase, and a heterologous amidophosphoribosyltransferase comprising a mutation either reducing or abolishing amidophosphoribosyltransferase protein activity. Any combination of two different forms of the amidophosphoribosyltransferase genes listed above are useful in the methods of the invention, with the limitation that at least one of the forms of the amidophosphoribosyltransferase has at least 10% of the activity of the polypeptide set forth in SEQ ID NO:3.

Thus, in one embodiment, the invention provides a method for identifying a test compound as a candidate for an antibiotic, comprising: providing an organism having a first form of an amidophosphoribosyltransferase; providing an organism having a second form of the amidophosphoribosyltransferase; and determining the growth of the organism having the first form of the amidophosphoribosyltransferase and the growth of the organism having the second form of the amidophosphoribosyltransferase in the presence of the test compound, wherein a difference in growth between the two organisms in the presence of the test compound indicates that the test compound is a candidate for an antibiotic. It is recognized in the art that the optional determination of the growth of the organism having the first form of the amidophosphoribosyltransferase and the growth of the organism having the second form of the amidophosphoribosyltransferase in the absence of any test compounds is performed to control for any inherent differences in growth as a result of the different genes. Growth and/or proliferation of an organism are measured by methods well known in the art such as optical density measurements, and the like. In a preferred embodiment, the organism is Magnaporthe grisea.

In another embodiment, the invention provides a method for identifying a test compound as a candidate for an antibiotic, comprising: providing an organism having a first form of an amidophosphoribosyltransferase; providing a comparison organism having a second form of the amidophosphoribosyltransferase; and determining the pathogenicity of the organism having the first form of the amidophosphoribosyltransferase and the organism having the second form of the amidophosphoribosyltransferase in the presence of the test compound, wherein a difference in pathogenicity between the two organisms in the presence of the test compound indicates that the test compound is a candidate for an antibiotic. In an optional embodiment of the inventon, the pathogenicity of the organism having the first form of the amidophosphoribosyltransferase and the organism having the second form of the amidophosphoribosyltransferase in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

In one embodiment of the invention, the first form of an amidophosphoribosyltransferase is SEQ ID NO:1 or SEQ ID NO:2, and the second form of the amidophosphoribosyltransferase is an amidophosphoribosyltransferase that confers a growth conditional phenotype (i.e. an adenine requiring phenotype) and/or a hypersensitivity or hyposensitivity phenotype on the organism. In a related embodiment of the invention, the second form of the amidophosphoribosyltransferase is SEQ ID NO:1 comprising a transposon insertion that reduces activity. In a related embodiment of the invention, the second form of an amidophosphoribosyltransferase is SEQ ID NO:1 comprising a transposon insertion that abolishes activity. In a related embodiment of the invention, the second form of the amidophosphoribosyltransferase is SEQ ID NO:2 comprising a transposon insertion that reduces activity. In a related embodiment of the invention, the second form of the amidophosphoribosyltransferase is SEQ ID NO:2 comprising a transposon insertion that abolishes activity. In a related embodiment of the invention, the second form of the amidophosphoribosyltransferase is Schizosaccharomyces pombe amidophosphoribosyltransferase. In a related embodiment of the invention, the second form of the amidophosphoribosyltransferase is Saccharomyces kluyveri amidophosphoribosyltransferase. In a related embodiment of the invention, the second form of the amidophosphoribosyltransferase is Saccharomyces cerevisiae amidophosphoribosyltransferase.

In another embodiment of the invention, the first form of an amidophosphoribosyltransferase is Schizosaccharomyces pombe amidophosphoribosyltransferase and the second form of the amidophosphoribosyltransferase is Schizosaccharomyces pombe amidophosphoribosyltransferase comprising a transposon insertion that reduces activity. In a related embodiment of the invention, the second form of the amidophosphoribosyltransferase is Schizosaccharomyces pombe amidophosphoribosyltransferase comprising a transposon insertion that abolishes activity. In another embodiment of the invention, the first form of an amidophosphoribosyltransferase is Saccharomyces kluyveri amidophosphoribosyltransferase and the second form of the amidophosphoribosyltransferase is Saccharomyces kluyveri amidophosphoribosyltransferase comprising a transposon insertion that reduces activity. In a related embodiment of the invention, the second form of the amidophosphoribosyltransferase is Saccharomyces kluyveri amidophosphoribosyltransferase comprising a transposon insertion that abolishes activity. In yet another embodiment of the invention, the first form of an amidophosphoribosyltransferase is Saccharomyces cerevisiae amidophosphoribosyltransferase and the second form of the amidophosphoribosyltransferase is Saccharomyces cerevisiae amidophosphoribosyltransferase comprising a transposon insertion that reduces activity. In a related embodiment of the invention, the second form of the amidophosphoribosyltransferase is Saccharomyces cerevisiae amidophosphoribosyltransferase comprising a transposon insertion that abolishes activity.

Conditional lethal mutants and/or antipathogenic mutants identify particular biochemical and/or genetic pathways given that at least one identified target gene is present in that pathway. Knowledge of these pathways allows for the screening of test compounds as candidates for antibiotics as inhibitors of the substrates, products, proteins and/or enzymes of the pathway. The invention provides methods of screening for an antibiotic by determining whether a test compound is active against the purine biosynthetic pathway on which amidophosphoribosyltransferase functions. Pathways known in the art are found at the Kyoto Encyclopedia of Genes and Genomes and in standard biochemistry texts (See, e.g. Lehninger et al., Principles of Biochemistry, New York, Worth Publishers (1993)).

Thus, in one embodiment, the invention provides a method for screening for test compounds acting against the biochemical and/or genetic pathway or pathways in which amidophosphoribosyltransferase functions, comprising: providing an organism having a first form of a gene in the purine biosynthetic pathway; providing an organism having a second form of the gene in the purine biosynthetic pathway; and determining the growth of the two organisms in the presence of a test compound, wherein a difference in growth between the organism having the first form of the gene and the organism having the second form of the gene in the presence of the test compound indicates that the test compound is a candidate for an antibiotic. It is recognized in the art that the optional determination of the growth of the organism having the first form of the gene and the organism having the second form of the gene in the absence of any test compounds is performed to control for any inherent differences in growth as a result of the different genes. Growth and/or proliferation of an organism are measured by methods well known in the art such as optical density measurements, and the like. In a preferred embodiment, the organism is Magnaporthe grisea.

The forms of a gene in the purine biosynthetic pathway useful in the methods of the invention include, for example, wild-type and mutated genes encoding phosphoribosylglycinamide formyltransferase and adenylosuccinate synthase from any organism, preferably from a fungal organism, and most preferrably from M. grisea. The forms of a mutated gene in the purine biosynthetic pathway comprise a mutation either reducing or abolishing protein activity. In one example, the form of a gene in the purine biosynthetic pathway comprises a transposon insertion. Any combination of a first form of a gene in the purine biosynthetic pathway and a second form of the gene listed above are useful in the methods of the invention, with the limitation that one of the forms of a gene in the purine biosynthetic pathway has at least 10% of the activity of the corresponding M. grisea gene.

In another embodiment, the invention provides a method for screening for test compounds acting against the biochemical and/or genetic pathway or pathways in which amidophosphoribosyltransferase functions, comprising: providing an organism having a first form of a gene in the purine biosynthetic pathway; providing an organism having a second form of the gene in the purine biosynthetic pathway; and determining the pathogenicity of the two organisms in the presence of the test compound, wherein a difference in pathogenicity between the organism having the first form of the gene and the organism having the second form of the gene in the presence of the test compound indicates that the test compound is a candidate for an antibiotic. In an optional embodiment of the inventon, the pathogenicity of the two organisms in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

Thus, in an alternate embodiment, the invention provides a method for screening for test compounds acting against the biochemical and/or genetic pathway or pathways in which amidophosphoribosyltransferase functions, comprising: providing paired growth media containing a test compound, wherein the paired growth media comprise a first medium and a second medium and the second medium contains a higher level of adenine than the first medium; inoculating the first and the second medium with an organism; and determining the growth of the organism, wherein a difference in growth of the organism between the first and the second medium indicates that the test compound is a candidate for an antibiotic. In one embodiment of the invention, the growth of the organism is determined in the first and the second medium in the absence of any test compounds to control for any inherent differences in growth as a result of the different media. Growth and/or proliferation of the organism are measured by methods well known in the art such as optical density measurements, and the like. In a preferred embodiment, the organism is Magnaporthe grisea.

One embodiment of the invention is directed to the use of multi-well plates for screening of antibiotic compounds. The use of multi-well plates is a format that readily accommodates multiple different assays to characterize various compounds, concentrations of compounds, and fungal organisms in varying combinations and formats. Certain testing parameters for the screening method can significantly affect the identification of growth inhibitors, and thus can be manipulated to optimize screening efficiency and/or reliability. Notable among these factors are variable sensitivities of different mutants, increasing hypersensitivity with increasingly less permissive conditions, an apparent increase in hypersensitivity with increasing compound concentration, and other factors known to those in the art.

### EXPERIMENTAL

### Example 1

### Construction of Plasmids with a Transposon Containing a Selectable Marker

Construction of Sif Transposon:

Sif was constructed using the GPS3 vector from the GPS-M mutagenesis system from New England Biolabs, Inc. (Beverly, Mass.) as a backbone. This system is based on the bacterial transposon Tn7. The following manipulations were done to GPS3 according to Sambrook et al., Molecular Cloning, a Laboratory Manual, Cold Spring Harbor Laboratory Press (1989). The kanamycin resistance gene (npt) contained between the Tn7 arms was removed by EcoRV digestion. The bacterial hygromycin B phosphotransferase (hph) gene (Gritz & Davies, 25 Gene 179 (1983) (PMID: 6319235)) under control of the Aspergillus nidulans trpC promoter and terminator (Mullaney et al., 199 Mol. Gen. Genet. 37 (1985) (PMID: 3158796)) was cloned by a HpaI/EcoRV blunt ligation into the Tn7 arms of the GPS3 vector yielding pSif1. Excision of the ampicillin resistance gene (bla) from pSif1 was achieved by cutting pSif1 with XmnI and BglI followed by a T4 DNA polymerase treatment to remove the 3′ overhangs left by the BglI digestion and religation of the plasmid to yield pSif. Top 10F′ electrocompetent E. coli cells (Invitrogen) were transformed with ligation mixture according to manufacturer's recommendations. Transformants containing the Sif transposon were selected on LB agar (Sambrook et al., supra) containing 50 μg/ml of hygromycin B (Sigma Chem. Co., St. Louis, Mo.).

### Example 2

### Construction of a Fungal Cosmid Library

Cosmid libraries were constructed in the pcosKA5 vector (Hamer et al., 98 Proc. Nat'l. Acad. Sci. USA 5110 (2001) (PMID: 11296265)) as described in Sambrook et al. Cosmid libraries were quality checked by pulsed-field gel electrophoresis, restriction digestion analysis, and PCR identification of single genes.

### Example 3

### Construction of Cosmids with Transposon Insertion into Fungal Genes

Sif Transposition into a Cosmid:

Transposition of Sif into the cosmid framework was carried out as described by the GPS-M mutagenesis system (New England Biolabs, Inc.). Briefly, 2 μl of the 10×GPS buffer, 70 ng of supercoiled pSIF, 8-12 μg of target cosmid DNA were mixed and taken to a final volume of 20 μl with water. 1 μl of transposase (TnsABC) was added to the reaction and incubated for 10 minutes at 37° C. to allow the assembly reaction to occur. After the assembly reaction, 1 μl of start solution was added to the tube, mixed well, and incubated for 1 hour at 37° C. followed by heat inactivation of the proteins at 75° C. for 10 minutes. Destruction of the remaining untransposed pSif was completed by PISceI digestion at 37° C. for 2 hours followed by a 10 minute incubation at 75° C. to inactivate the proteins. Transformation of Top 10F′ electrocompetent cells (Invitrogen) was done according to manufacturers recommendations. Sif-containing cosmid transformants were selected by growth on LB agar plates containing 50 μg/ml of hygromycin B (Sigma Chem. Co.) and 100 μg/ml of Ampicillin (Sigma Chem. Co.).

### Example 4

### High Throughput Preparation and Verification of Transposon Insertion into the M. grisea ADE4 Gene

E. coli strains containing cosmids with transposon insertions were picked to 96 well growth blocks (Beckman Co.) containing 1.5 ml of TB (Terrific Broth, Sambrook et al., supra) supplemented with 50 μg/ml of ampicillin. Blocks were incubated with shaking at 37° C. overnight. E. coli cells were pelleted by centrifugation and cosmids were isolated by a modified alkaline lysis method (Marra et al., 7 Genome Res. 1072 (1997) (PMID: 9371743)). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

The DNA sequences adjacent to the site of the transposon insertion were used to search DNA and protein databases using the BLAST algorithms (Altschul et al., supra). A single insertion of SIF into the Magnaporthe grisea ADE4 gene was chosen for further analysis. This construct was designated cpgmra0023017e01 and it contains the SIF transposon insertion approximately between amino acids 300 and 301 relative to the Schizosaccharomyces pombe homolog.

### Example 5

### Preparation of ADE4 Cosmid DNA and Transformation of Magnaporthe grisea

Cosmid DNA from the ADE4 transposon tagged cosmid clone was prepared using QIAGEN Plasmid Maxi Kit (Qiagen), and digested by PI-PspI (New England Biolabs, Inc.). Fungal electro-transformation was performed essentially as described (Wu et al., 10 MPMI 700 (1997)). Briefly, M. grisea strain Guy 11 was grown in complete liquid media (Talbot et al., 5 Plant Cell 1575 (1993) (PMID: 8312740)) shaking at 120 rpm for 3 days at 25° C. in the dark. Mycelia was harvested and washed with sterile H₂O and digested with 4 mg/ml beta-glucanase (InterSpex) for 4-6 hours to generate protoplasts. Protoplasts were collected by centrifugation and resuspended in 20% sucrose at a concentration of 2×10⁸ protoplasts/ml. 50 μl of protoplast suspension was mixed with 10-20 μg of the cosmid DNA and pulsed using a Gene Pulser II instrument (BioRad) set with the following parameters: 200 ohm, 25 μF, and 0.6 kV. Transformed protoplasts were regenerated in complete agar media (Talbot et al., supra) with the addition of 20% sucrose for one day, then overlayed with CM agar media containing hygromycin B (250 ug/ml) to select transformants. Transformants were screened for homologous recombination events in the target gene by PCR (Hamer et al., supra). Two independent strains were identified and are hereby referred to as K1-4 and K1-8, respectively.

### Example 6

### Effect of Transposon Insertion on Magnaporthe Pathogenicity

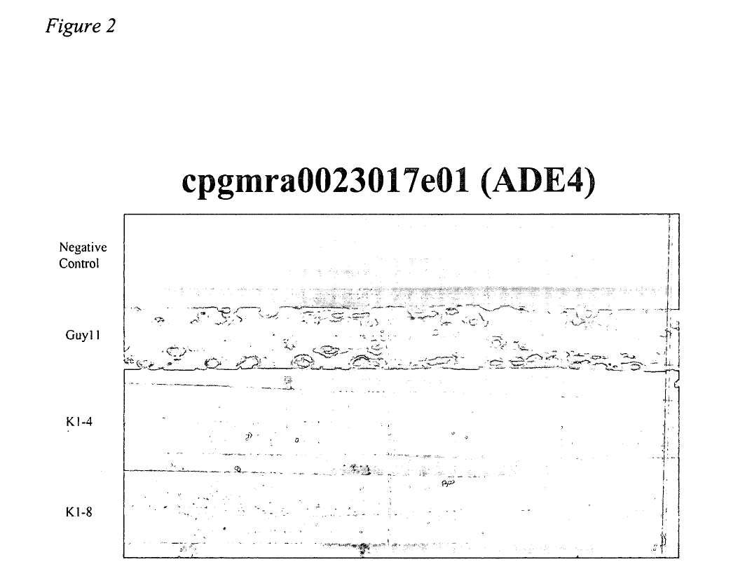

The target fungal strains, K1-4 and K1-8, obtained in Example 5 and the wild-type strain, Guy11, were subjected to a pathogenicity assay to observe infection over a 1-week period. Rice infection assays were performed using Indian rice cultivar CO₃₉ essentially as described in Valent et al. (Valent et al., 127 Genetics 87 (1991) (PMID: 2016048)). All three strains were grown for spore production on complete agar media. Spores were harvested and the concentration of spores adjusted for whole plant inoculations. Two-week-old seedlings of cultivar CO39 were sprayed with 12 ml of conidial suspension (5×10⁴ conidia per ml in 0.01% Tween-20 solution). The inoculated plants were incubated in a dew chamber at 27° C. in the dark for 36 hours, and transferred to a growth chamber (27° C. 12 hours/21° C. 12 hours at 70% humidity) for an additional 5.5 days. Leaf samples were taken at 3, 5, and 7 days post-inoculation and examined for signs of successful infection (i.e. lesions). FIG. 2 shows the effects of ADE4 gene disruption on Magnaporthe infection at seven days post-inoculation.

### Example 7

### Verification of ADE4 Gene Function by Analysis of Nutritional Requirements

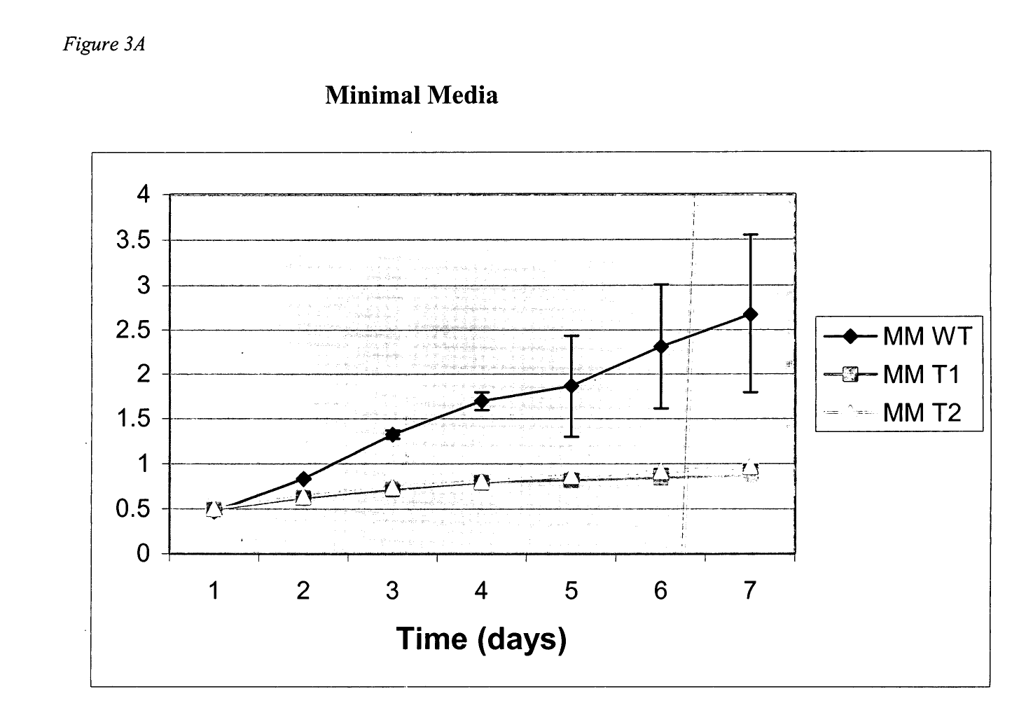

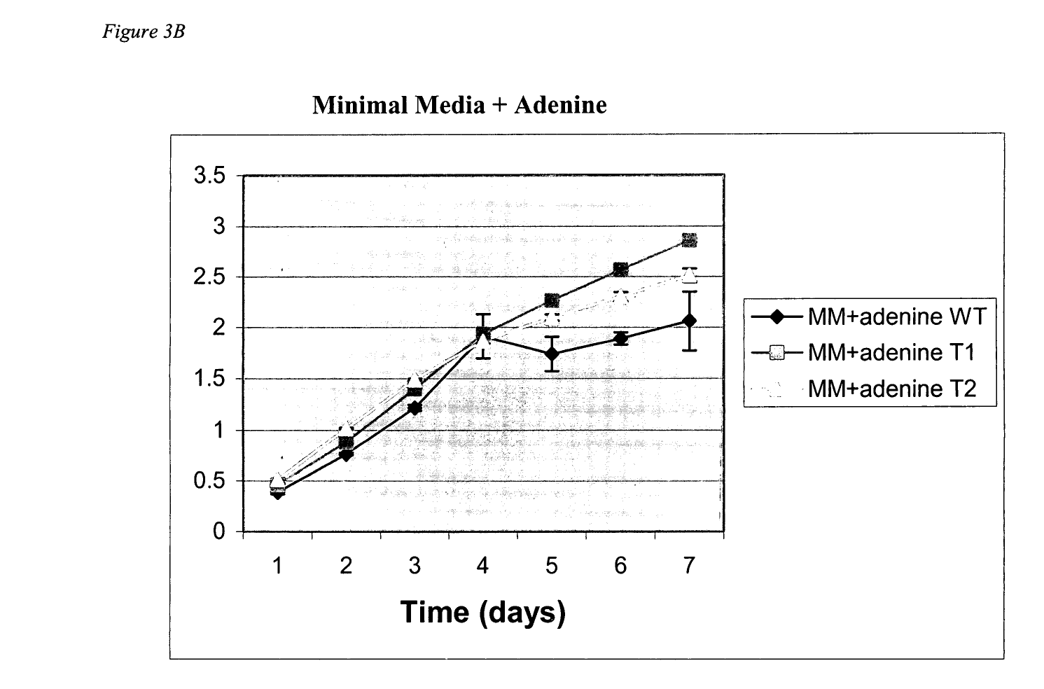

The fungal strains, K1-4 and K1-8, containing the ADE4 disrupted gene obtained in Example 5 were analyzed for their nutritional requirement for adenine using the PM5 phenotype microarray from Biolog, Inc. (Hayward, Calif.). The PM5 plate tests for the auxotrophic requirement for 94 different metabolites. The inoculating fluid consists of 0.05% Phytagel, 0.03% Pluronic F68, 1% glucose, 23.5 mM NaNO₃, 6.7 mM KCl, 3.5 mM Na₂SO₄, 11.0 mM KH₂PO₄, 0.01% p-iodonitrotetrazolium violet, 0.1 mM MgCl₂, 1.0 mM CaCl₂ and trace elements, pH adjusted to 6.0 with NaOH. Final concentrations of trace elements are: 7.6 μM ZnCl₂, 2.5 μM MnCl₂.4H₂O, 1.8 μM FeCl₂.4H₂O, 0.71 μM CoCl₂.6H₂O, 0.64 μM CuCl₂.2H₂O, 0.62 μM Na₂MoO₄, 18 μM H₃BO₃. Spores for each strain were harvested into the inoculating fluid. The spore concentrations were adjusted to 2×10⁵ spores/ml. 100 μl of spore suspension were deposited into each well of the microtiter plates. The plates were incubated at 25° C. for 7 days. Optical density (OD) measurements at 490 nm and 750nm were taken daily. The OD₄₉₀ measures the extent of tetrazolium dye reduction and the level of growth, and OD₇₅₀ measures growth only. Turbidity=OD₄₉₀+OD₇₅₀. Data confirming the annotated gene function is presented as a graph of Turbidity vs. Time showing both the mutant fungi and the wild-type control in the absence and presence of adenine (FIGS. 3A and 3B).

### Example 8

### Cloning, Expression, and Purification of Amidophosphoribosyltransferase Protein

The following is a protocol to obtain a purified amidophosphoribosyltransferase protein.

Cloning and Expression Strategies:

An amidophosphoribosyltransferase cDNA gene is cloned into E. coli (PET vectors-Novagen), Baculovirus (Pharmingen) and Yeast (Invitrogen) expression vectors containing His/fusion protein tags, and the expression of recombinant protein is evaluated by SDS-PAGE and Western blot analysis.

Extraction:

Extract recombinant protein from 250 ml cell pellet in 3 ml of extraction buffer by sonicating 6 times, with 6 second pulses at 4° C. Centrifuge extract at 15000×g for 10 minutes and collect supernatant. Assess biological activity of the recombinant protein by activity assay.

Purification:

Purify recombinant protein by Ni-NTA affinity chromatography (Qiagen). Purification protocol (perform all steps at 4° C.):
Use 3 ml Ni-beads
   Equilibrate column with the buffer
   Load protein extract
   Wash with the equilibration buffer
   Elute bound protein with 0.5 M imidazole

Another method for purifying amidophosphoribosyltransferase protein is described in Tso et al. (1982) J Biol Chem 257:3532-3536.

### Example 9

### Assays for Measuring Binding of Test Compounds to Amidophosphoribosyltransferase

The following is a protocol to identify test compounds that bind to the amidophosphoribosyltransferase protein.
Purified full-length amidophosphoribosyltransferase polypeptide with a His/fusion protein tag (Example 8) is bound to a HISGRAB Nickel Coated Plate (Pierce, Rockford, Ill.) following manufacturer's instructions.
Buffer conditions are optimized (e.g. ionic strength or pH, Shoolingin-Jordan et al. (1997) Methods Enzymol 281: 309-16 (PMID: 9250995)) for binding of radiolabeled 5-phospho-beta-D-ribosylamine, diphosphate, L-glutamate, 5-phospho-alpha-D-ribose 1-diphosphate, or L-glutamine to the bound amidophosphoribosyltransferase protein.
Screening of test compounds is performed by adding test compound and radioactive 5-phospho-beta-D-ribosylamine, diphosphate, L-glutamate, 5-phospho-alpha-D-ribose 1-diphosphate, or L-glutamine to the wells of the HISGRAB plate containing bound amidophosphoribosyltransferase protein.
The wells are washed to remove excess labeled ligand and scintillation fluid (SCINTIVERSE, Fisher Scientific) is added to each well.
The plates are read in a microplate scintillation counter.
Candidate compounds are identified as wells with lower radioactivity as compared to control wells with no test compound added.

Additionally, a purified polypeptide comprising 10-50 amino acids from the M. grisea amidophosphoribosyltransferase is screened in the same way. A polypeptide comprising 10-50 amino acids is generated by subcloning a portion of the ADE4 gene into a protein expression vector that adds a His-Tag when expressed (see Example 8). Oligonucleotide primers are designed to amplify a portion of the ADE4 gene using the polymerase chain reaction amplification method. The DNA fragment encoding a polypeptide of 10-50 amino acids is cloned into an expression vector, expressed in a host organism and purified as described in Example 8 above.

Test compounds that bind amidophosphoribosyltransferase are further tested for antibiotic activity. M. grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

Test compounds that bind amidophosphoribosyltransferase are further tested for antipathogenic activity. M. grisea is grown as described for spore production on oatmeal agar media (Talbot et al., supra). Spores are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO₃₉ essentially as described in Valent et al., supra). Two-week-old seedlings of cultivar CO₃₉ are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity as compared to the control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

### Example 10

### Assays for Testing Inhibitors or Candidates for Inhibition of Amidophosphoribosyltransferase Activity

The enzymatic activity of amidophosphoribosyltransferase is determined in the presence and absence of candidate compounds in a suitable reaction mixture, such as described by Tso et al. (1982) supra. Candidate compounds are identified by a decrease in products or a lack of a decrease in substrates in the presence of the compound, with the reaction proceeding in either direction.

Candidate compounds are additionally determined in the same manner using a polypeptide comprising a fragment of the M. grisea amidophosphoribosyltransferase. The amidophosphoribosyltransferase polypeptide fragment is generated by subcloning a portion of the ADE4 gene into a protein expression vector that adds a His-Tag when expressed (see Example 8). Oligonucleotide primers are designed to amplify a portion of the ADE4 gene using polymerase chain reaction amplification method. The DNA fragment encoding the amidophosphoribosyltransferase polypeptide fragment is cloned into an expression vector, expressed and purified as described in Example 8 above.

Test compounds identified as inhibitors of amidophosphoribosyltransferase activity are further tested for antibiotic activity and antipathogenic activity as described in Example 9.

### Example 11

### Assays for Testing Compounds for Alteration of Amidophosphoribosyltransferase Gene Expression

Magnaporthe grisea fungal cells are grown under standard fungal growth conditions that are well known and described in the art. Wild-type M. grisea spores are harvested from cultures grown on complete agar or oatmeal agar media after growth for 10-13 days in the light at 25° C. using a moistened cotton swab. The concentration of spores is determined using a hemacytometer and spore suspensions are prepared in a minimal growth medium to a concentration of 2×10⁵ spores per ml. 25 ml cultures are prepared to which test compounds will be added at various concentrations. A culture with no test compound present is included as a control. The cultures are incubated at 25° C. for 3 days after which test compound or solvent only control is added. The cultures are incubated an additional 18 hours. Fungal mycelia is harvested by filtration through Miracloth (CalBiochem, La Jolla, Calif.), washed with water, and frozen in liquid nitrogen. Total RNA is extracted with TRIZOL Reagent using the methods provided by the manufacturer (Life Technologies, Rockville, Md.). Expression is analyzed by Northern analysis of the RNA samples as described (Sambrook et al., supra) using a radiolabeled fragment of the ADE4 gene as a probe. Test compounds resulting in an altered level of ADE4 mRNA relative to the untreated control sample are identified as candidate antibiotic compounds.

Test compounds identified as inhibitors of amidophosphoribosyltransferase activity are further tested for antibiotic activity and antipathogenic activity as described in Example 9.

### Example 12

### In Vivo Cell Based Assay Screening Protocol with a Fungal Strain Containing a Mutant Form of Amidophosphoribosyltransferase that Lacks Activity

The effect of test compounds on the growth of wild-type fungal cells and mutant fungal cells having a mutant ADE4 gene is measured and compared as follows. Magnaporthe grisea fungal cells containing a mutant form of the ADE4 gene that lacks activity, for example a ADE4 gene containing a transposon insertion, are grown under standard fungal growth conditions that are well known and described in the art. Magnaporthe grisea spores are harvested from cultures grown on complete agar medium containing adenine (Sigma) after growth for 10-13 days in the light at 25° C. using a moistened cotton swab. The concentration of spores is determined using a hemacytometer and spore suspensions are prepared in a minimal growth medium containing adenine to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild-type cells are screened under the same conditions.

The effect of each of the test compounds on the mutant and wild-type fungal cells is measured against the growth control and the percent of inhibition is calculated as the OD₅₉₀ (fungal strain plus test compound)/OD₅₉₀ (growth control)×100. The percent of growth inhibition in the presence of the test compound on the mutant and wild-type fungal strains are compared. Compounds that show differential growth inhibition between the mutant and the wild-type cells are identified as potential antifungal compounds. Similar protocols may be found in Kirsch & DiDomenico, 26 Biotechnology 177 (1994) (PMID: 7749303)).

Test compounds that produce a differential growth response between the mutant and wild-type fungal strains are further tested for antipathogenic activity as described in Example 9.

### Example 13

### In Vivo Cell Based Assay Screening Protocol with a Fungal Strain Containing a Mutant Form of Amidophosphoribosyltransferase with Reduced Activity

The effect of test compounds on the growth of wild-type fungal cells and mutant fungal cells having a mutant ADE4 gene is measured and compared as follows. Magnaporthe grisea fungal cells containing a mutant form of the ADE4 gene resulting in reduced activity, such as a the transposon insertion mutation of cpgmra0023017e01 or a promoter truncation mutation that reduces expression, are grown under standard fungal growth conditions that are well known and described in the art. A promoter truncation is made by deleting a portion of the promoter upstream of the transcription start site using standard molecular biology techniques that are well known and described in the art (Sambrook et al., supra).

The mutant and wild-type Magnaporthe grisea spores are harvested from cultures grown on complete agar medium containing adenine (Sigma) after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild-type cells are screened under the same conditions.

The effect of each test compound on the mutant and wild-type fungal strains is measured against the growth control and the percent of inhibition is calculated as the OD₅₉₀ (fungal strain plus test compound)/OD₅₉₀ (growth control)×100. The percent growth inhibition as a result of each of the test compounds on the mutant and wild-type cells is compared. Compounds that show differential growth inhibition between the mutant and the wild-type cells are identified as potential antifungal compounds. Similar protocols may be found in Kirsch & DiDomenico, supra.

Test compounds that produce a differential growth response between the mutant and wild-type fungal strains are further tested for antipathogenic activity as described in Example 9.

### Example 14

### In Vivo Cell Based Assay Screening Protocol with a Fungal Strain Containing a Mutant Form of a Purine Biosynthetic Gene that Lacks Activity

The effect of test compounds on the growth of wild-type fungal cells and mutant fungal cells having a mutant form of a gene in the purine biosynthetic pathway is measured and compared as follows. Magnaporthe grisea fungal cells containing a mutant form of a gene that lacks activity in the purine biosynthetic pathway (e.g. phosphoribosylglycinamide formyltransferase having a transposon insertion) are grown under standard fungal growth conditions that are well known and described in the art. Magnaporthe grisea spores are harvested from cultures grown on complete agar medium containing adenine (Sigma) after growth for 10-13 days in the light at 25° C. using a moistened cotton swab. The concentration of spores is determined using a hemacytometer and spore suspensions are prepared in a minimal growth medium containing adenine to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild-type cells are screened under the same conditions.

The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the mutant and the wild-type cells are compared. Compounds that show differential growth inhibition between the mutant and the wild-type cells are identified as potential antifungal compounds. Similar protocols may be found in Kirsch & DiDomenico, supra.

Test compounds that produce a differential growth response between the mutant and wild-type fungal strains are further tested for antipathogenic activity as described in Example 9.

### Example 15

### In Vivo Cell Based Assay Screening Protocol with a Fungal Strain Containing a Mutant Form of a Purine Biosynthetic Gene with Reduced Activity

The effect of test compounds on the growth of wild-type fungal cells and mutant fungal cells having a mutant form of a gene in the purine biosynthetic pathway is measured and compared as follows. Magnaporthe grisea fungal cells containing a mutant form of a gene resulting in reduced protein activity in the purine biosynthetic pathway (e.g. phosphoribosylglycinamide formyltransferase having a promoter truncation that reduces expression), are grown under standard fungal growth conditions that are well known and described in the art. Mutant and wild-type Magnaporthe grisea spores are harvested from cultures grown on complete agar medium containing adenine (Sigma) after growth for 10-13 days in the light at 25° C. using a moistened cotton swab. The concentration of spores is determined using a hemacytometer and spore suspensions are prepared in a minimal growth medium to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild-type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the mutant and wild-type cells are compared. Compounds that show differential growth inhibition between the mutant and the wild-type cells are identified as potential antifungal compounds. Similar protocols may be found in Kirsch & DiDomenico, supra.

Test compounds that produce a differential growth response between the mutant and wild-type fungal strains are further tested for antipathogenic activity as described in Example 9.

### Example 16

### In Vivo Cell Based Assay Screening Protocol with a Fungal Strain Containing a Heterologous Amidophosphoribosyltransferase Gene

The effect of test compounds on the growth of wild-type fungal cells and fungal cells lacking a functional endogenous amidophosphoribosyltransferase gene and containing a heterologous amidophosphoribosyltransferase gene is measured and compared as follows. Wild-type M. grisea fungal cells and M. grisea fungal cells lacking an endogenous amidophosphoribosyltransferase gene and containing a heterologous amidophosphoribosyltransferase gene from Schizosaccharomyces pombe (Genbank Accession No. P41390), having 53% sequence identity, are grown under standard fungal growth conditions that are well known and described in the art.

A M. grisea strain carrying a heterologous amidophosphoribosyltransferase gene is made as follows. A M. grisea strain is made with a nonfunctional endogenous amidophosphoribosyltransferase gene, such as one containing a transposon insertion in the native gene that abolishes protein activity. A construct containing a heterologous amidophosphoribosyltransferase gene is made by cloning a heterologous amidophosphoribosyltransferase gene, such as from Schizosaccharomyces pombe, into a fungal expression vector containing a trpC promoter and terminator (e.g. Carroll et al., 41 Fungal Gen. News Lett. 22 (1994) (describing pCB1003) using standard molecular biology techniques that are well known and described in the art (Sambrook et al., supra). The vector construct is used to transform the M. grisea strain lacking a functional endogenous amidophosphoribosyltransferase gene. Fungal transformants containing a functional amidophosphoribosyltransferase gene are selected on minimal agar medium lacking adenine, as only transformants carrying a functional amidophosphoribosyltransferase gene grow in the absence of adenine.

Wild-type strains of M. grisea and strains containing a heterologous form of amidophosphoribosyltransferase are grown under standard fungal growth conditions that are well known and described in the art. M. grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. The effect of each compound on the wild-type and heterologous fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the wild-type and heterologous fungal strains are compared. Compounds that show differential growth inhibition between the wild-type and heterologous strains are identified as potential antifungal compounds with specificity to the native or heterologous amidophosphoribosyltransferase gene products. Similar protocols may be found in Kirsch & DiDomenico, supra.

Test compounds that produce a differential growth response between the strain containing a heterologous gene and strain containing a fungal gene are further tested for antipathogenic activity as described in Example 9.

### Example 17

### Pathway Specific In Vivo Assay Screening Protocol

Compounds are tested as candidate antibiotics as follows. Magnaporthe grisea fungal cells are grown under standard fungal growth conditions that are well known and described in the art. Wild-type M. grisea spores are harvested from cultures grown on oatmeal agar media after growth for 10-13 days in the light at 25° C. using a moistened cotton swab. The concentration of spores is determined using a hemocytometer and spore suspensions are prepared in a minimal growth medium and a minimal growth medium containing adenine (Sigma) to a concentration of 2×10⁵ spores per ml. The minimal growth media contains carbon, nitrogen, phosphate, and sulfate sources, and magnesium, calcium, and trace elements (for example, see inoculating fluid in Example 7). Spore suspensions are added to each well of a 96-well microtiter plate (approximately 4×10⁴ spores/well). For each well containing a spore suspension in minimal media, an additional well is present containing a spore suspension in minimal medium containing adenine.

Test compounds are added to wells containing spores in minimal media and minimal media containing adenine. The total volume in each well is 200 μl. Both minimal media and adenine containing media wells with no test compound are provided as controls. The plates are incubated at 25° C. for seven days and optical density measurements at 590 nm are taken daily. A compound is identified as a candidate for an antibiotic acting against the adenine biosynthetic pathway when the observed growth in the well containing minimal media is less than the observed growth in the well containing adenine as a result of the addition of the test compound. Similar protocols may be found in Kirsch & DiDomenico, supra.

Published references and patent publications cited herein are incorporated by reference as if terms incorporating the same were provided upon each occurrence of the individual reference or patent document. While the foregoing describes certain embodiments of the invention, it will be understood by those skilled in the art that variations and modifications may be made that will fall within the scope of the invention. The foregoing examples are intended to exemplify various specific embodiments of the invention and do not limit its scope in any manner.

1. A method for identifying a test compound as a candidate for an antibiotic, comprising:
a) contacting an amidophosphoribosyltransferase polypeptide with a test compound; and
b) detecting the presence or absence of binding between the test compound and the amidophosphoribosyltransferase polypeptide, wherein binding indicates that the test compound is a candidate for an antibiotic.

2. The method of claim 1, wherein the amidophosphoribosyltransferase polypeptide is selected from the group consisting of: a fungal amidophosphoribosyltransferase polypeptide, a Magnaporthe amidophosphoribosyltransferase polypeptide, and SEQ ID NO:3.

3. A method for identifying a test compound as a candidate for an antibiotic, comprising:
a) contacting a test compound with a polypeptide selected from the group consisting of:
i) a polypeptide consisting essentially of SEQ ID NO:3;
ii) a polypeptide having at least ten consecutive amino acids of SEQ ID NO:3;
iii) a polypeptide having at least 50% sequence identity with SEQ ID NO:3 and at least 10% of the activity of SEQ ID NO:3; and
iv) a polypeptide consisting of at least 50 amino acids having at least 50% sequence identity with SEQ ID NO:3 and at least 10% of the activity of SEQ ID NO:3; and
b) detecting the presence and/or absence of binding between the test compound and the polypeptide, wherein binding indicates that the test compound is a candidate for an antibiotic.

4. A method for identifying a test compound as a candidate for an antibiotic, comprising:
a) contacting 5-phospho-beta-D-ribosylamine, diphosphate, and L-glutamate with an amidophosphoribosyltransferase in the presence and absence of a test compound or contacting 5-phospho-alpha-D-ribose 1-diphosphate and L-glutamine with an amidophosphoribosyltransferase in the presence and absence of a test compound; and
b) determining a concentration for at least one of 5-phospho-beta-D-ribosylamine, diphosphate, L-glutamate, 5-phospho-alpha-D-ribose 1-diphosphate and/or L-glutamine in the presence and absence of the test compound, wherein a change in the concentration for any of 5-phospho-beta-D-ribosylamine, diphosphate, L-glutamate, 5-phospho-alpha-D-ribose 1-diphosphate and/or L-glutamine indicates that the test compound is a candidate for an antibiotic.

5. The method of claim 4, wherein the amidophosphoribosyltransferase is selected from the group consisting of a fungal amidophosphoribosyltransferase, a Magnaporthe amidophosphoribosyltransferase, and SEQ ID NO:3.

6. A method for identifying a test compound as a candidate for an antibiotic, comprising:
a) contacting an amidophosphoribosyltransferase polypeptide with 5-phospho-beta-D-ribosylamine, diphosphate, and L-glutamate in the presence and absence of a test compound or with 5-phospho-alpha-D-ribose 1-diphosphate and L-glutamine in the presence and absence of a test compound, wherein the amidophosphoribosyltransferase polypeptide is selected from the group consisting of:
i) a polypeptide having at least 50% sequence identity with SEQ ID NO:3 and at least 10% of the activity of SEQ ID NO:3,
ii) a polypeptide consisting essentially of SEQ ID NO:3,
iii) a polypeptide comprising at least 50 consecutive amino acids of SEQ ID NO:3 and having at least 10% of the activity of SEQ ID NO:3; and
iv) a polypeptide consisting of at least 50 amino acids having at least 50% sequence identity with SEQ ID NO:3 and having at least 10% of the activity of SEQ ID NO:3; and
b) determining a concentration for at least one of 5-phospho-beta-D-ribosylamine, diphosphate, L-glutamate, 5-phospho-alpha-D-ribose 1-diphosphate and/or L-glutamine in the presence and absence of the test compound, wherein a change in the concentration for any of 5-phospho-beta-D-ribosylamine, diphosphate, L-glutamate, 5-phospho-alpha-D-ribose 1-diphosphate and/or L-glutamine indicates that the test compound is a candidate for an antibiotic.

7. A method for identifying a test compound as a candidate for an antibiotic, comprising:
a) measuring the expression of an amidophosphoribosyltransferase in an organism, or a cell or tissue thereof, in the presence and absence of a test compound; and
b) comparing the expression of the amidophosphoribosyltransferase in the presence and absence of the test compound, wherein an altered expression in the presence of the test compound indicates that the test compound is a candidate for an antibiotic.

8. The method of claim 7, wherein the organism is a fungus or the organism is a Magnaporthe fungus.

9. The method of claim 7, wherein the amidophosphoribosyltransferase is SEQ ID NO:3.

10. The method of claim 7, wherein the expression of the amidophosphoribosyltransferase is measured by detecting the amidophosphoribosyltransferase mRNA, or the amidophosphoribosyltransferase polypeptide, or the amidophosphoribosyltransferase polypeptide enzyme activity.

11. A method for identifying a test compound as a candidate for an antibiotic, comprising:
a) providing a fungal organism having a first form of an amidophosphoribosyltransferase;
b) providing a fungal organism having a second form of the amidophosphoribosyltransferase, wherein one of the first or the second form of the amidophosphoribosyltransferase has at least 10% of the activity of SEQ ID NO:3; and
c) determining the growth of the organism having the first form of the amidophosphoribosyltransferase and the organism having the second form of the amidophosphoribosyltransferase in the presence of a test compound,
wherein a difference in growth between the two organisms in the presence of the test compound indicates that the test compound is a candidate for an antibiotic.

12. The method of claim 11, wherein the fungal organism having the first form of the amidophosphoribosyltransferase and the fungal organism having the second form of the amidophosphoribosyltransferase are Magnaporthe and the first and the second form of the amidophosphoribosyltransferase are fungal amidophosphoribosyltransferases.

13. The method of claim 11, wherein the first form of the amidophosphoribosyltransferase is SEQ ID NO:1 or SEQ ID NO:2.

14. The method of claim 11, wherein the fungal organism having the first form of the amidophosphoribosyltransferase and the fungal organism having the second form of the amidophosphoribosyltransferase are Magnaporthe and the first form of the amidophosphoribosyltransferase is SEQ ID NO:1 or SEQ ID NO:2.

15. The method of claim 11, wherein the fungal organism having the first form of the amidophosphoribosyltransferase and the fungal organism having the second form of the amidophosphoribosyltransferase are Magnaporthe, the first form of the amidophosphoribosyltransferase is SEQ ID NO:1 or SEQ ID NO:2, and the second form of the amidophosphoribosyltransferase is a heterologous amidophosphoribosyltransferase.

16. The method of claim 11, wherein the fungal organism having the first form of the amidophosphoribosyltransferase and the fungal organism having the second form of the amidophosphoribosyltransferase are Magnaporthe, the first form of the amidophosphoribosyltransferase is SEQ ID NO:1 or SEQ ID NO:2, and the second form of the amidophosphoribosyltransferase is SEQ ID NO:1 or SEQ ID NO:2 comprising a transposon insertion that reduces or abolishes amidophosphoribosyltransferase activity.

17. A method for identifying a test compound as a candidate for an antibiotic, comprising:
a) providing a fungal organism having a first form of an amidophosphoribosyltransferase;
b) providing a fungal organism having a second form of the amidophosphoribosyltransferase, wherein one of the first or the second form of the amidophosphoribosyltransferase has at least 10% of the activity of SEQ ID NO:3; and
c) determining the pathogenicity of the organism having the first form of the amidophosphoribosyltransferase and the organism having the second form of a amidophosphoribosyltransferase in the presence of a test compound,
wherein a difference in pathogenicity between the two organisms in the presence of the test compound indicates that the test compound is a candidate for an antibiotic.

18. The method of claim 17, wherein the fungal organism having the first form of the amidophosphoribosyltransferase and the fungal organism having the second form of the amidophosphoribosyltransferase are Magnaporthe and the first and the second form of the amidophosphoribosyltransferase are fungal amidophosphoribosyltransferases.

19. The method of claim 17, wherein the first form of the amidophosphoribosyltransferase is SEQ ID NO:1 or SEQ ID NO:2.

20. The method of claim 17, wherein the fungal organism having the first form of the amidophosphoribosyltransferase and the fungal organism having the second form of the amidophosphoribosyltransferase are Magnaporthe and the first form of the amidophosphoribosyltransferase is SEQ ID NO:1 or SEQ ID NO:2.

21. The method of claim 17, wherein the fungal organism having the first form of the amidophosphoribosyltransferase and the fungal organism having the second form of the amidophosphoribosyltransferase are Magnaporthe, the first form of the amidophosphoribosyltransferase is SEQ ID NO:1 or SEQ ID NO:2, and the second form of the amidophosphoribosyltransferase is a heterologous amidophosphoribosyltransferase.

22. The method of claim 17, wherein the fungal organism having the first form of the amidophosphoribosyltransferase and the fungal organism having the second form of the amidophosphoribosyltransferase are Magnaporthe, the first form of the amidophosphoribosyltransferase is SEQ ID NO:1 or SEQ ID NO:2, and the second form of the amidophosphoribosyltransferase is SEQ ID NO:1 or SEQ ID NO:2 comprising a transposon insertion that reduces or abolishes amidophosphoribosyltransferase activity.

23. A method for identifying a test compound as a candidate for an antibiotic, comprising:
a) providing a fungal organism having a first form of a gene in the purine biosynthetic pathway;
b) providing a fungal organism having a second form of said gene in the purine biosynthetic pathway, wherein one of the first or the second form of the gene has at least 10% of the activity of a corresponding Magnaportha grisea gene; and
c) determining the growth of the organism having the first form of the gene and the organism having the second form of the gene in the presence of a test compound,
wherein a difference in growth between the two organisms in the presence of the test compound indicates that the test compound is a candidate for an antibiotic.

24. The method of claim 23, wherein the fungal organism having the first form of the gene and the fungal organism having the second form of the gene are Magnaporthe.

25. The method of claim 23, wherein the fungal organism having the first form of the gene and the fungal organism having the second form of the gene are Magnaporthe, the first form of the gene in the purine biosynthetic pathway is Magnaporthe grisea phosphoribosylglycinamide formyltransferase, and the second form of the gene is a heterologousphosphoribosylglycinamide formyltransferase.

26. The method of claim 23, wherein the fungal organism having the first form of the gene and the fungal organism having the second form of the gene are Magnaporthe, the first form of the gene in the purine biosynthetic pathway is Magnaporthe grisea phosphoribosylglycinamide formyltransferase, and the second form of the gene is Magnaporthe grisea phosphoribosylglycinamide formyltransferase comprising a transposon insertion that reduces or abolishes phosphoribosylglycinamide formyltransferase protein activity.

27. The method of claim 23, wherein the fungal organism having the first form of the gene and the fungal organism having the second form of the gene are Magnaporthe, the first form of the gene in the purine biosynthetic pathway is Magnaporthe grisea adenylosuccinate synthase, and the second form of the gene is a heterologous adenylosuccinate synthase.

28. The method of claim 23, wherein the fungal organism having the first form of the gene and the fungal organism having the second form of the gene are Magnaporthe, the first form of the gene in the purine biosynthetic pathway is Magnaporthe grisea adenylosuccinate synthase, and the second form of the gene is Magnaporthe grisea adenylosuccinate synthase comprising a transposon insertion that reduces or abolishes adenylosuccinate synthase protein activity.

29. A method for identifying a test compound as a candidate for an antibiotic, comprising:
a) providing a fungal organism having a first form of a gene in the purine biosynthetic pathway;
b) providing a fungal organism having a second form of said gene in the purine biosynthetic pathway, wherein one of the first or the second form of the gene has at least 10% of the activity of a corresponding Magnaportha grisea gene; and
c) determining the pathogenicity of the organism having the first form of the gene and the organism having the second form of the gene in the presence of a test compound,
wherein a difference in pathogenicity between the organism and the comparison organism in the presence of the test compound indicates that the test compound is a candidate for an antibiotic.

30. The method of claim 29, wherein the fungal organism having the first form of the gene and the fungal organism having the second form of the gene are Magnaporthe.

31. The method of claim 29, wherein the fungal organism having the first form of the gene and the fungal organism having the second form of the gene are Magnaporthe, the first form of the gene in the purine biosynthetic pathway is Magnaporthe grisea phosphoribosylglycinamide formyltransferase, and the second form of the gene is a heterologous phosphoribosylglycinamide formyltransferase.

32. The method of claim 29, wherein the fungal organism having the first form of the gene and the fungal organism having the second form of the gene are Magnaporthe, the first form of the gene in the purine biosynthetic pathway is Magnaporthe grisea phosphoribosylglycinamide formyltransferase, and the second form of the gene is Magnaporthe grisea phosphoribosylglycinamide formyltransferase comprising a transposon insertion that reduces or abolishes phosphoribosylglycinamide formyltransferase protein activity.

33. The method of claim 29, wherein the fungal organism having the first form of the gene and the fungal organism having the second form of the gene are Magnaporthe, the first form of the gene in the purine biosynthetic pathway is Magnaporthe grisea adenylosuccinate synthase, and the second form of the gene is a heterologous adenylosuccinate synthase.

34. The method of claim 29, wherein the fungal organism having the first form of the gene and the fungal organism having the second form of the gene are Magnaporthe, the first form of a gene in the purine biosynthetic pathway is Magnaporthe grisea adenylosuccinate synthase, and the second form of the gene is Magnaporthe grisea adenylosuccinate synthase comprising a transposon insertion that reduces or abolishes adenylosuccinate synthase protein activity.

35. A method for identifying a test compound as a candidate for an antibiotic, comprising:
a) providing paired growth media containing a test compound, wherein the paired growth media comprise a first medium and a second medium and the second medium contains a higher level of adenine than the first medium;
b) inoculating the first and the second medium with an organism; and
c) determining the growth of the organism, wherein a difference in growth of the organism between the first and second medium indicates that the test compound is a candidate for an antibiotic.

36. The method of claim 35, wherein the organism is a fungus or where the organism is a Magnaporthe fungus.


SEQUENCE LISTING (5 sequences)

SEQ ID NO: 1 - DNA, 1614 bp, Magnaporthe grisea
atgtgtggtgtatccgcgctccttttgggcgacacaaaagcccagtcagccgctgtcgacctgcacgagtctctgtactttcttcagcaccgtgggcaagatgcggccggtatcgccgtctgccaaggcggcagggtctatcagtgcaagggcaacggcatggccgccaaggtgttcgacgagggccgaaagacggccgacctaccgggcttcatgggaatcgcccacctgagataccccaccgctggaacttcgtcggcgtccgaagcccagcctttctacgtgaactccccgttcggtctctccatgagcgtcaacggaaaccttgtcaacagccctgagctcatcagcttcttggaccgcgaggctcgccgccacgtaaacaccgactcggattcggaactgctgctcaacgtctttgctcatgccctttacgagctgaacaaggcccgagcaaacgtggacgatgtgtttaccgctcttcgcgaggtttatgcgaggtgccacggcgcatttgcgtgcactgctatgatcgcaggatttggcatcctgggtttccgtgaccaaaacggcatccgcccgctttgcctaggctcccgcccgtcgacaactctggagggcgctacagactacttcctggcttccgaatcggtctcgctgacccagctgggcttcagcaacattgtggacattcttcccggacaggccgtcttcatccgcaagaacggtacccccgagttccgccagatcgtggagaggaagtcctacaccccagacatctttgaatttgtttactttgccaggcccgacagccacattgacggagtgtcggtacaccgcagccggcagaacatgggcttgaagttggccaagaagatgagggaaacgcttagcgaacaggcaatcaaggagattgacgtcataatcccggttcccgagacaagtaatacctcggcagctgtactggctacagagctgaacaagcccttctcgaacgcattcgtcaagaaccgctacgtctacaggacatttatcctccctggtcagcaggcccgacaaaagagcgttcgccgcaaactttctccgatcgcatccgagttcaacggcaaggttgtatgcttggtggacgattcgattgtgaggggaactacctcgcgcgagattgtccagatggcgagggaggccggtgcaaccaaggttcttttcgtctcatgtagtcccgaagtgacgcatcctcacgtccacggtatcgaccttgccgacccggctgagcttattgctcatggaaagacaagggaggaagtcgccacgctcatcgacgcagacgaggttgtgtatcagtctcttgacgatctcaaggccgcatgttttgatgcagccggacccaacaacgacgtcaatgattttgaggtcggtgtcttttgtggcaagtacaagacagaggttccggaaggctattttgagcacctcagccgcgtccagggcgccaagaagaaggccaaggctgttgcaaatgttgccagcagcgggccaacaaatgatagccgaaacggactaatgagccctgaacatcgggaagacatcagtcttcacaacttcgcaaccaaaccttga

SEQ ID NO: 2 - DNA, 2357 bp, Magnaporthe grisea
atgtgtggtgtatccgcgctccttgtaaggccatgacccctccccctccaaaaaataaaagaaaaaaaaaggcccgacgcgcctcgcgcaaaaaaagagcgcgccacaaggcgcgaatacccaacgaaaagcaacttcaacgctaacctttgcatgtcttgagcggaattagttgggcgacacaaaagcccagtcagccgctgtcgacctgcacgagtctctgtactttcttcagcacgtcagtaaaccccccgacggcgatcggcctgtcatcccgtgaaattttgtggcagtctatcatggacaggagggcggttcctgcagatggcgtctttttgcccaacatttaatggaacaagagcatgttcactgacgctgggaatggatagcgtgggcaagatgcggccggtatcgccgtctgccaaggcggcagggtctatcagtgcaagggcaacggcatggccgccaaggtgttcgacgagggccgaaagacggccgacctaccgggcttcatgggaatcgcccacctgagataccccaccgctggaacttcgtcggcgtacgtgctgccatgtcctttgttgtccccgtcaaaggcccagctctcgaggatgttggcgttcctgacacaaccggcactgtgcacaggtccgaagcccagcctttctacgtgaactccccgttcggtctctccatgagcgtcaacggaaaccttgtcaacagccctgagctcatcagcttcttggaccgcgaggctcgccgccacgtaaacaccgactcggattcggaactgctgtaggtcttggaatgacgtatgatactgatcaaggaatgggattatgctgaccaggtttctactgcacccaggctcaacgtctttgctcatgccctttacgagctgaacaaggcccgagcaaacgtggacgatgtgtttaccgctcttcgcgaggtttatgcgaggtgccacggcgcatttgcgtgcactgctatgatcgcaggatttggcatcctgggtttccggtaattgctgcctatattctggctcgtaactgaagcttacagcacctcggttctaacaagctaccccttacagtgaccaaaacggcatccgcccgctttgcctaggctcccgcccgtcgacaactctggagggcgctacagactacttcctggcttccgaatcggtctcgctgacccagctgggcttcagcaacattgtggacattcttcccggacaggccgtcttcatccgcaagaacggtacccccgagttccgccagatcgtggagaggaagtcctacaccccagacatctttgaatttgtttactttgccaggcccgacagccacattgacggagtgtcggtacaccgcagccggcagaacatgggcttgaagttggccaagaagatgagggaaacgcttagcgaacaggcaatcaaggagattgacgtcagtaagttacaccccggttcttctgatcaactcggcgccgagtagattctaatcctggactctttttacagtaatcccggttcccgaggtgcgcaagtattccggacattgtcggcttttgctggcggcaccgggatggactacgattactgacttagcctcatgtgttagacaagtaatacctcggcagctgtactggctacagagctgaacaagcccttctcgaacgcattcgtcaagaaccgctacgtctacaggacatttatcctccctggtcagcaggcccgacaaaagagcgttcgccgcaaactttctccgatcgcatccgagttcaacggcaaggttgtatgcttggtggacgattcgattgtgaggggaactacctcgcgcgagattgtccagatggcgagggaggccggtgcaaccaaggttcttttcgtctcatgtagtcccgaagtgacgcatcctcacgtccacggtatcgaccttgccgacccggctgagcttattgctcatggaaagacaagggaggaagtcgccacgctcatcgacgcagacgaggttgtgtatcagtctcttgacgatctcaaggccgcatgttttgatgcagccggacccaacaacgacgtcaatgattttgaggtcggtgtcttttgtggcaagtacaagacagaggttccggaaggctattttgagcacctcagccgcgtccagggcgccaagaagaaggccaaggctgttgcaaatgttgccagcagcgggccaacaaatgatagccgaaacggactaatgagccctgaacatcgggaagacatcaggtaagccttcaaaccctctttgctcgaatgcaaagattgtgactaacacctctatagtcttcacaacttcgcaaccaaaccttga

SEQ ID NO: 3 - protein, 537 aa, Magnaporthe grisea
MCGVSALLLGDTKAQSAAVDLHESLYFLQHRGQDAAGIAVCQGGRVYQCKGNGMAAKVFDEGRKTADLPGFMGIAHLRYPTAGTSSASEAQPFYVNSPFGLSMSVNGNLVNSPELISFLDREARRHVNTDSDSELLLNVFAHALYELNKARANVDDVFTALREVYARCHGAFACTAMIAGFGILGFRDQNGIRPLCLGSRPSTTLEGATDYFLASESVSLTQLGFSNIVDILPGQAVFIRKNGTPEFRQIVERKSYTPDIFEFVYFARPDSHIDGVSVHRSRQNMGLKLAKKMRETLSEQAIKEIDVIIPVPETSNTSAAVLATELNKPFSNAFVKNRYVYRTFILPGQQARQKSVRRKLSPIASEFNGKVVCLVDDSIVRGTTSREIVQMAREAGATKVLFVSCSPEVTHPHVHGIDLADPAELIAHGKTREEVATLIDADEVVYQSLDDLKAACFDAAGPNNDVNDFEVGVFCGKYKTEVPEGYFEHLSRVQGAKKKAKAVANVASSGPTNDSRNGLMSPEHREDISLHNFATKP

SEQ ID NO: 4 - DNA, 2826 bp, Magnaportha grisea
atgtcgggagagggacaaacggcgacggcgccggcgccggcgcctgcatcgcaggccaacaatggcgcgaaggagcagacgcagggacctcaggcagactcgcaagtcaagacatcagggtcgccggcgacggcaaactccaacattccaaccaaccagccgggacatccgagcttccgaagacaaagagcatctagagcttgcgagacttgtcacgcaagaaagatagaatgtcgcattccctcaccgaaaagaaagaagacacatgctacccaatcagccacacaatccagagattctgacaggagtcaaagtgagcgcgagcgcgaggccgacgatgatccaactccgcctgccaacagcgggcctgctgccccaaacttcacgcgcccggccgcagtctttcatacgagcgaagggacccccaacactgcgatgggagacgaacaggccaagaaggtagaagtcgacaggaataaatatgtcgacatgctggtgaggccgcaattcacaagagcgcccatcaaggatgctggcagggttgcattcctgggagagtcttcaaacctcacgctgctggtgcacgataggcagggctccgactccgatgtggttcattaccccttgccggaaaatgtaaaggggtcgagggcaaggatgaccgagctggacaacgtcgagattgacatcttgcaccaacgtggcgccttcctcctgccgcccaggtcactatgcgacgaactcattgatgcttatttcaagtggatacacccgatagttcccgtcatcaaccgcacacatttcatgaaccaatacaacgaccccaagaatccaccgtcactgcttttactccaagccatactgctcgcaggctctagggtctgcacaaacccggcgttgatggatgccaatggctcatccacccctgcagctttgacattctacaagagagcaaaggctctctacgacgccaactacgaagacgacagggtgactctcgttcaggctctcctcctcatgggctggtactgggagggccctgaggatgtgaccaagaacgtcttctactggactcgggttgctactgtggttgctgagggctcaggcatgcacaggagtgtagagtcgtctcagttgagtcgggccgacaagaagctgtggaaacgcatttggtggactcttttcacgcgcgatcgctcagttgctgtagccctcggccgaccagtccacataaacctcgacgactctgacgtggagatgttgacagaggaggacttcaacgaggacgagcccggtcttccaagccaatatccgccagatcaaagacatgtgcagttttttctgcaatatgttaagctctgtgaaatcatgggccttgtgctatcccaacagtattcggtagcatcgaaaggccgacaaagaaacccgatcgacttgacacatagtgatatggcgctggcggattggttacaaaactgccccaagattgtttactgggagatgccgaggcatcacttctggtctgcattgctgcactccaactactacacgacactatgccttctccacagggcacacatgccgccgagtgggtatcggaataaattccccgaggattcagcatatccgtcacgcaatattgctttccaggcggcggcgatgattacctccatcatcgagaacctgtctgcacacgatcaactgcggtactgtcccgcctttatcgtttacagcttgttctcggcacttattatgcacgtctaccagatgaagtccccagtgccaacgattcagcaagtaacacaggacaggattcggacatgtatgcaagcactgaaggacgtttcgcgtgtttggctcgtcggaaaaatggtctggacgctgttccaatccatcttaggcaacaaggttctggaggagaggcttcagaaggctgcaggtaaacgacacaggaaagcgcaacaaatcctgaacaggctcgatcagcatgccgcgcagcagcaacacgagcaaaactctcatcagccatcactccacgaggctaagcggaagtatgacgagatggcgattgacttcaacaacacgccacaaccccaagaatcatatgtgcgatcgcggccccagaccccgagcatgtcagcgaggcatgaaactgctggaggcggccacatgaatggcaacaatggcgctatgccaccacctctgacgtccccaaatgcgcggctggatgctttcatgggaggaaccggatcgcacccgacgacgaggcctgcgacgccattcaacccatccatgtcgatgccgacaactcccccagatttgtacttggtcaccaggaactcacccaacctatcgcagtcaatatgggagaactttcagcccgatcaactattcccagagagtgcacatatgccggccttcccaccccagatgtcaccacagcagacgcaccaaaatgtcgaccccagcatgatgcagttcaaccaaagttcgcagactggggagaatcacactgcaccgggaacatcgtccgagtcattcggagcgcagataaaaacaagcggcagcccactgcagaacagcggcagtccggtccagtttggccagatgtcgggcaacgggttacctgctggattctgggccaatttggatacgacggctggtcctattcaggacggtcaaagccccgacagctgggggagtgcatcatctgcccatggtggtcctgcggtcccgtctacgctcaatgtggaagactggctacaattctttggtatcaatggaaacggcgaaaacctgaatatcgacttttctgcacttgtttaa

SEQ ID NO: 5 - protein, 941 aa, Magnaportha grisea
MSGEGQTATAPAPAPASQANNGAKEQTQGPQADSQVKTSGSPATANSNIPTNQPGHPSFRRQRASRACETCHARKIECRIPSPKRKKTHATQSATQSRDSDRSQSEREREADDDPTPPANSGPAAPNFTRPAAVFHTSEGTPNTAMGDEQAKKVEVDRNKYVDMLVRPQFTRAPIKDAGRVAFLGESSNLTLLVHDRQGSDSDVVHYPLPENVKGSRARMTELDNVEIDILHQRGAFLLPPRSLCDELIDAYFKWIHPIVPVINRTHFMNQYNDPKNPPSLLLLQAILLAGSRVCTNPALMDANGSSTPAALTFYKRAKALYDANYEDDRVTLVQALLLMGWYWEGPEDVTKNVFYWTRVATVVAEGSGMHRSVESSQLSRADKKLWKRIWWTLFTRDRSVAVALGRPVHINLDDSDVEMLTEEDFNEDEPGLPSQYPPDQRHVQFFLQYVKLCEIMGLVLSQQYSVASKGRQRNPIDLTHSDMALADWLQNCPKIVYWEMPRHHFWSALLHSNYYTTLCLLHRAHMPPSGYRNKFPEDSAYPSRNIAFQAAAMITSIIENLSAHDQLRYCPAFIVYSLFSALIMHVYQMKSPVPTIQQVTQDRIRTCMQALKDVSRVWLVGKMVWTLFQSILGNKVLEERLQKAAGKRHRKAQQILNRLDQHAAQQQHEQNSHQPSLHEAKRKYDEMAIDFNNTPQPQESYVRSRPQTPSMSARHETAGGGHMNGNNGAMPPPLTSPNARLDAFMGGTGSHPTTRPATPFNPSMSMPTTPPDLYLVTRNSPNLSQSIWENFQPDQLFPESAHMPAFPPQMSPQQTHQNVDPSMMQFNQSSQTGENHTAPGTSSESFGAQIKTSGSPLQNSGSPVQFGQMSGNGLPAGFWANLDTTAGPIQDGQSPDSWGSASSAHGGPAVPSTLNVEDWLQFFGINGNGENLNIDFSALV